(19) United States
(12) Patent Application Publication
(10) Pub. No.: US 2014/0186265 A1
(43) Pub. Date: Jul. 3, 2014

(54) MULTIFUNCTIONAL BACTERIOPHAGE FOR DELIVERY OF THERAPEUTIC AGENTS AND IMAGING REAGENTS

(71) Applicant: Colorado State University Research Foundation, Fort Collins, CO (US)

(72) Inventors: Brian R. MCNAUGHTON, Fort Collins, CO (US); Sandra M. DEPORTER, Fort Collins, CO (US); Irene LUI, Fort Collins, CO (US); Virginia J. BRUCE, Fort Collins, CO (US)

(21) Appl. No.: 14/088,812

(22) Filed: Nov. 25, 2013

Related U.S. Application Data
(60) Provisional application No. 61/730,365, filed on Nov. 27, 2012.
(60) Provisional application No. 61/860,814, filed on Jul. 31, 2013.

(51) Int. Cl. C12N 7/00; C12N 15/10; C07K 14/005; C12N 15/86; A61K 47/48

(52) U.S. Cl. CPC C12N 7/00; C12N 15/86; A61K 47/48776; C07K 14/005; C12N 15/1037
USPC 424/9.1; 435/235.1; 435/320.1; 435/375; 435/456; 424/93.2; 435/5; 530/350; 506/9

(57) ABSTRACT

This invention relates to modified bacteriophage useful for the delivery of macromolecular biopolymers and nanoparticles to target cells, e.g., disease cells, and their use in cell-selective identification and imaging, as well as the treatment and prevention of diseases and other medical conditions.

### CROSS-REFERENCE TO RELATED APPLICATIONS

This application claims priority to U.S. Provisional Patent Application Ser. No. 61/730,365 filed Nov. 27, 2012 and U.S. Provisional Patent Application Ser. No. 61/860,814 filed Jul. 31, 2013. These applications are incorporated herein by reference in their entireties.

### STATEMENT REGARDING GOVERNMENT INTEREST

This invention was made with government support under Grant No. W81XWH-10-1-0182 awarded by the Army Medical Research and Material Command. The government has certain rights in the invention.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is CSUV_{—}002_{—}02US_ST25.txt. The text file is 8 KB, was created on Nov. 25, 2013, and is being submitted electronically via EFS-Web.

### FIELD OF THE INVENTION

This invention relates to modified bacteriophage useful for the delivery of macromolecular biopolymers and nanoparticles to target cells, e.g., disease cells, and microorganisms, e.g., viruses, and their use in cell-selective or microorganism-specific identification and imaging, as well as the treatment and prevention of diseases, infections and other medical conditions.

### BACKGROUND OF THE INVENTION

Macromolecular biopolymers (also referred to herein as macromolecules) and nanoparticles offer unique opportunities for advances in medicine. Recent studies suggest that small molecules (<500 Da) are capable of regulating only a small portion of the human proteome. Moreover, the functional diversity of proteins successfully targeted by small molecules is quite low; for example, 40% of all prescription drugs target the G-protein coupled receptors. Unlike most of their small molecule counterparts, macromolecules have high folding energies (typically ˜7-20 kcal/mol) that allow them to adopt large, precise three-dimensional surfaces suitable for strongly binding to a cellular target in a manner that completely abrogates or alters its function. The size and complexity of folded macromolecules can result in specificities of action that are not easily achievable using small molecules. In view of these and other advantages associated with macromolecular biopolymers, there is a strong need and a compelling opportunity to address barriers that impede the development of macromolecule therapeutic agents.

Perhaps the most important impediment to the broader use of macromolecular agents is the difficulties associated with their delivery across the lipid bilayer membrane of mammalian cells into the interior of mammalian cells. In addition to the delivery of macromolecular or nanoscale reagents to the inside of mammalian cells, the targeted delivery of macromolecule therapeutic reagents to diseased cells is ideal. Targeted delivery of toxic therapeutics directly and selectively to diseased cells lessens or abrogates the potentially deleterious off-target effects that may be associated with treating healthy cells. In addition, disease cell-selective imaging is needed to accurately identify disease cells from a complex solution, and to potentially assist in the surgical removal of diseased cells. Furthermore, actively localizing imaging reagents to the interior of a diseased cell effectively locks that signal to the internalized cell, thus increasing the signal to noise ratio and simplifying the isolation, identification, or localization of diseased cells.

Most commonly, targeted delivery of cargo is achieved by conjugating the cargo to an antibody; fragment antigen-binding region (Fab fragment), nucleic acid aptamer, or peptide reagent that binds a cell surface receptor overexpressed on a targeted cell as compared to non-targeted cells. Antibody-based methods (e.g., immunoconjugates) are by far the most common approach; however, serious medical, practical, and financial barriers limit their more general use. Perhaps the most common of these limitations are antibody or fragment immunogenicity, normal tissue expression of the targeted antigen, unreliable antigen expression on diseased tissue, inefficient delivery across the cell membrane (typically >5%), inefficient release of functional drug to the cytoplasm of the targeted cell, and long circulation times (typically weeks), which result in high background activity for extended periods of time. Mass production, distribution, and storage of whole antibodies and their functional fragments are difficult and expensive. Preparing immunoconjugate reagents often requires chemical ligation and purification steps, which increases the complexity and cost of their production. These molecules are unstable over long periods of time (weeks to months), and typically require storage conditions that include aqueous buffers and relatively low temperatures (−20-4° C.). As a result of these severe limitations, their use in medical applications is challenging, both from a production and economic standpoint.

Clearly, there remains a need in the art for new and more effective means to deliver macromolecular biopolymers and nanoparticles to diseased cells, in order to identify such cells and treat their disease. Similarly, there is a need in the art for new and more effective means to deliver or bind macromolecular biopolymers and nanoparticles to other targets, including other cells and microorganisms, including infectious agents, e.g., viruses, for a variety of uses, including the diagnosis, prognosis, treatment and prevention of diseases and infections.

### BRIEF SUMMARY OF THE INVENTION

The present invention includes novel peptide transduction domains (PTDs) and multifunctional phage, e.g., for the delivery of therapeutic agents and imaging reagents, as well as related methods of producing and using the same.

In one aspect, the invention includes an isolated peptide comprising or consisting of an amino acid sequence having at least 90%, at least 95%, at least 98%, or at least 99% identity to the amino acid sequence set forth in SEQ ID NO:1. In certain embodiments, the peptide comprises or consists of the amino acid sequence set forth in any one of SEQ ID NOs:1-32. In other embodiments, the peptide comprises or consists of two or more amino acid sequences having at least 90%, at least 95%, at least 98%, or at least 99% identity to the amino acid sequence set forth in SEQ ID NO:1. In particular embodiments, the peptide comprises or consists of two or more amino acid sequences having the sequence set forth in any one of SEQ ID NOs: 1-32.

In another embodiment, the invention includes a fusion polypeptide comprising an isolated peptide of the invention and a bacteriophage coat protein or portion thereof. In certain embodiments, the bacteriophage coat protein is selected from: p3, p6, p7, p8 and p9. In one embodiment, the bacteriophage coat protein is p3.

In a related embodiment, the invention includes an isolated polynucleotide comprising a sequence encoding a peptide or fusion polypeptide of the invention.

In a further related embodiment, the invention includes a vector comprising a polynucleotide of the invention.

In another embodiment, the invention includes a bacteriophage comprising a peptide fusion polypeptide, polynucleotide or of the invention. In particular embodiments, the bacteriophage is an M13 bacteriophage.

In another aspect, the invention includes a genetically modified bacteriophage, said bacteriophage comprising: a targeting agent that binds to and/or mediates transduction of a target cell or microorganism, wherein the targeting agent is displayed on the phage surface; and one or more conjugation peptide displayed on the phage surface. In certain embodiments, the targeting agent is fused to a bacteriophage coat protein, bacteriophage coat protein is selected from the group consisting of: p3, p6, p7, p8 and p9. In particular embodiments, the target cell is a disease cell, such as a tumor cell, e.g., a prostate cancer cell. In particular embodiments, the microorganism is a virus, bacterium, fungus, algae or protozoan. In certain embodiments, the targeting agent is a peptide, e.g., a PTD, a protein, an antibody or fragment thereof, a nanobody, or a nucleic acid. In certain embodiments, the one or more conjugation peptide comprises one or more sortase bridging domain. In one embodiment, the one or more sortase bridging domain comprises a sortase A bridging domain or a sortase B bridging domain, wherein the sortase A bridging domain optionally comprises two or more glycine residues, and where the sortase B bridging domain optionally comprises two or more alanine residues. In other embodiments, the one or more conjugation peptide is fused to one or more phage coat protein, such as, e.g., p8 or p9. In one embodiment, the genetically modified bacteriophage comprises a first conjugation peptide fused to a first phage coat protein and a second conjugation peptide fused to a second phage coat protein, e.g., p8 and p9.

In certain embodiments of the genetically modified bacteriophage, a therapeutic macromolecular biopolymer, an imaging reagent, or a peptide that binds an imaging reagent is conjugated to one of said one or more conjugation peptides. In certain embodiments, an endosomolytic peptide is conjugated to one of said one or more conjugation peptides. In particular embodiments, the therapeutic macromolecular biopolymer, imaging reagent, or peptide that binds an imaging reagent is conjugated to p8 or p9.

In another related aspect, the invention includes a genetically modified bacteriophage, said bacteriophage comprising: a targeting agent, e.g., a peptide, that binds to and/or mediates transduction of a target cell or microorganism, wherein said targeting agent is displayed on the phage surface; and a therapeutic macromolecular biopolymer, an imaging reagent, or a peptide that binds an imaging reagent, wherein the therapeutic macromolecular biopolymer, the imaging reagent, or the peptide that binds an imaging reagent is displayed on the phage surface. In particular embodiments, the targeting agent is fused to a bacteriophage coat protein. In certain embodiments, the bacteriophage coat protein is selected from: p3, p6, p7, p8 and p9.

In particular embodiments of the various aspects of the invention, the target cell is a disease cell, e.g., tumor cell, such as a prostate cancer cell. In particular embodiments, the microorganism is a virus.

In certain embodiments, the targeting agent is any of the peptides described herein.

In certain embodiments, the bacteriophage further comprises a therapeutic macromolecular biopolymer, imaging reagent, or peptide that binds an imaging reagent, wherein the therapeutic macromolecular biopolymer, imaging reagent, or peptide that binds an imaging reagent is coupled to a phage coat protein or portion thereof on said bacteriophage. In particular embodiments, the therapeutic macromolecular biopolymer, imaging reagent, or peptide that binds an imaging reagent is coupled to the phage coat protein or portion thereof via a protease-cleavable linker. In other embodiments, the therapeutic macromolecular biopolymer is conjugated to the phage coat protein or portion thereof via a conjugation peptide. In certain embodiments, the conjugation peptide is a sortase bridging domain. In particular embodiments, the bacteriophage comprises a therapeutic macromolecular biopolymer and further comprises: an endosomolytic peptide, wherein the endosomolytic peptide is displayed on the phage surface. In one embodiment, the endosomolytic peptide is conjugated to a phage coat protein or portion thereof on said bacteriophage, such as, e.g., p3, p6, p7, p8 or p9. In one embodiment, the phage coat protein is p9. In particular embodiments, the endosomolytic peptide is conjugated to the phage coat protein or portion thereof via a conjugation peptide. In certain embodiments, the bacteriophage comprises the protease-cleavable linker and an endosomolytic peptide. In particular embodiments, the peptide that binds the imaging agent is an iron oxide nanoparticle-binding peptide. In certain embodiments, the imaging agent comprises iron oxide nanoparticles.

In another aspect, the invention includes a method of delivering a therapeutic macromolecular biopolymer or an imaging reagent to a cell, said method comprising contacting said cell with the bacteriophage of the invention, wherein said bacteriophage comprises pr is conjugated to a therapeutic macromolecular biopolymer or an imaging agent. In particular embodiments, the method is performed in vitro, in vivo or ex vivo.

In a related embodiments, the invention includes a method of delivering a therapeutic macromolecular biopolymer or an imaging reagent to a subject in need thereof, comprising providing to said subject a bacteriophage of the invention, wherein said bacteriophage comprises pr is conjugated to a therapeutic macromolecular biopolymer or an imaging agent. In particular embodiments, the subject has been diagnosed with or is considered at risk of having a disease. In one embodiment, the disease is a tumor, e.g., a prostate tumor. In certain embodiments, the bacteriophage is provided to the subject parenterally, orally, or by inhalation. In particular embodiments wherein an imaging agent is delivered to the subject, the method further comprises detecting the location of the imaging agent within the subject.

In another aspect, the invention includes a method of determining the presence of or an amount of a target cell within a plurality of cells, comprising contacting the plurality of cells with a bacteriophage of the invention, wherein the bacteriophage comprises a coupled or bound imaging reagent, washing the bacteriophage from the plurality of cells, and then detecting the presence of or the amount of the imaging agent within the plurality of cells, thus determining the presence of or the amount of target cell within the plurality of cells.

In a further related aspect, the invention includes a method of treating or preventing a disease or infection in a subject in need thereof, comprising providing to the subject an effective amount of the bacteriophage of the invention, wherein said bacteriophage comprises a coupled therapeutic macromolecular polymer. In certain embodiments, the disease is a tumor, e.g., a prostate cancer. In certain embodiments, the infection is a bacterial infection or a viral infection. In particular embodiments, the bacteriophage is provided to the subject parenterally.

In another aspect, the present invention includes a method of producing a genetically modified bacteriophage capable of delivering a therapeutic macromolecular polymer or an imaging agent to a target cell, comprising: identifying a bacteriophage that express on its surface a targeting peptide that mediates transduction of a target cell on the bacteriophage surface, or genetically modifying a bacteriophage to express on its surface a targeting peptide that mediates transduction of a target cell on the bacteriophage surface; and genetically modifying the bacteriophage to express one or more conjugation peptides on the phage surface. In certain embodiments, the one or more conjugation peptide is fused to one or more phage coat protein. In certain embodiments, the one or more phage coat protein is selected from: p3, p7, p8, and p9. In one embodiment, the one or more phage coat protein comprises p8 or p9. In one embodiment, a first conjugation peptide is fused to p8 and a second conjugation peptide is fused to p9. In certain embodiments, the first step comprises screening a plurality of bacteriophage to identify a bacteriophage capable of transducing the target cell. In particular embodiments, the plurality of bacteriophage is a library of genetically modified bacteriophage that express different exogenous peptides on the phage surface. In other embodiments, the method further comprises coupling said therapeutic macromolecular polymer, said imaging agent, or said peptide that binds an imaging agent to said genetically modified bacteriophage. In particular embodiments, the therapeutic macromolecular polymer, imaging agent, or peptide that binds an imaging agent comprises a coupling domain. In particular embodiments, the coupling domain is a sortase coupling domain. In particular embodiments, the coupling comprises contacting the genetically modified bacteriophage with the therapeutic macromolecular polymer, the imaging agent, or the peptide that binds an imaging agent, in the presence of a coupling agent. In certain embodiments, the one or more conjugation peptide comprises a sortase recognition domain, the coupling domain comprises a sortase coupling domain, and the coupling agent is a sortase. In certain embodiments, the one or more conjugation peptide comprises a sortase coupling domain, the coupling domain comprises a sortase recognition domain, and the coupling agent is a sortase.

### BRIEF DESCRIPTION OF THE DRAWINGS

FIG. 1 depicts multifunctional phage designed as targeted delivery vehicles for either biopolymer therapeutic reagents (A) or imaging agents, e.g., iron oxide nanoparticle MRI imaging agents (B).

FIG. 2 depicts a procedure for the selection of phage displaying a cell-selective protein transduction domain. The diagram shows the evolution of a PC-3 prostate cancer cell-selective protein transduction domain. Phage are incubated with PC-3 prostate cancer cells enriched for cell penetration. Cell-penetrating phage are then incubated with a polyanionic tissue culture plate, and phage that do not bind the tissue culture plate are enriched.

FIG. 3 provides graphs and fluorescence microscopy images showing internalized GFP in PC-3 cells after treatment with the indicated amounts of Ypep-GFP or Ypep-GFP-Ypep. (A) and (C) show flow cytometry data for Ypep-GFP (A) and Ypep-GFP-Ypep (C). (B) and (D) show fluorescent microscopy images of PC-3 cells after a 1 hour treatment with 10 Ypep-GFP (B) or Ypep-GFP-Ypep (D) and subsequent washing with PBS/20 U/mL heparin sulfate. Images were taken using a 500 msec, exposure.

FIG. 4 provides bar graphs showing the cell selectivity of: (A) Yep-GFP uptake; (B) Ypep-GFP-Ypep uptake; and (C) (Ypep)₅-phage uptake, as measured by mean cell fluorescence following administration of the indicated amounts of Yep-GFP or Ypep-GFP-Ypep, or 1.7 pM (Ypep)₅-phage to PC-3, LNCaP, HEK 293T, Hs 697.Sp, or MRC-9 cells. Ypep-GFP and Ypep-GFP-Ypep uptake was measured by flow cytometry, and (Ypep)₅-phage uptake levels were measured by titering for phage plaques in each mammalian cell lysate following treatment and washing. For FIGS. 4A and 4B, the legend from top to bottom corresponds to the bars from left to right for each cell type.

FIG. 5 provides fluorescence microscopy images of cells treated with 15 Ypep-GFP (A-F) or 1 μM Ypep-GFP-Ypep (G-L), at either 37° C. (A and G) or 4° C. (B and H), or after pre-treatment with 5 μg/mL filipin (C and I), 5 μg/mL cytochalasin D (D and J), 10 μg/mL chloropromazine (E and K), or 400 μg/mL heparin sulfate (F and L). Other conditions were identical for each treatment. All images were obtained using a 200 msec, exposure. Scale bars in each image=50 μm.

FIG. 6 provides a table showing the tissue distribution of (Ypep)₅-phage in mice. Mice were treated with 5 nM (Ypep)₅-phage, and tissues were harvested four hours later. Cells were washed and lysed, and cell lysates were titered for phage levels.

FIG. 7 provides fluorescence microscopy images of various cell lines treated with 1 mg/mL polyacrylamide nanoparticles conjugated to both GFP and (Ypep)₅-phage, including PC-3 prostate cancer cells (A), PNT-2 non-cancer protstate cells (B), HEK-293 non-cancer kidney cells (C), MRC-9 non-cancer lung cells (D), and Hs 697.Sp non-cancer spleen cells (E). Only targeted PC-3 cells were delivered large quantities of the GFP-embedded (Ypep)₅-phage conjugated nanoparticles.

FIG. 8 provides bar graphs showing the uptake efficiency of Ypep mutants having the indicated amino acid substitutions as compared to the wild-type Ypep amino acid sequence shown in (A). The fold-change in GFP uptake relative to wild-type Ypep is shown for alanine mutants of Ypep-GFP (B), Ypep-GFP mutants at residue 4 (C), Ypep-GFP mutants at residue 7 (D), and Ypep-GFP double mutants at residues 4 and 7 (E). In each example, PC-3 cells were treated with 5 μM mutant Ypep-GFP, then washed to remove cell surface-bound protein. Values and error bars represent the mean and standard deviation of three independent experiments.

FIG. 9 provides live cell fluorescence microscopy images of PC-3 cells following treatment with 5 μM of the indicated mutant Ypep-GP fusions, and washing to remove cell surface-bound protein. Green (lighter) color represents internalized GFP. The scale bar is 50 μm. Lamp intensity was set at 50%, with a 250 msec exposure for all images.

FIG. 10 provides a bar graph depicting flow cytometry data showing the amounts of GFP delivered to PC-3 cells following treatment with 1 μm of the indicated Ypep(mutant)-GFP fusions, Tat-GFP fusion, or penetrating-GFP fusion, and then washing to remove cell surface bound protein. Values and error bars represent the mean and standard deviation of three independent experiments.

FIG. 11 provides line graphs depicting flow cytometry date showing the amount of internalized GFP in PC-3 cells or HEK-293 cells following treatment with 0.1, 0.25, 0.5 or 1 of the indicated Ypep-GFP or Ypep(mutant)-GFP fusions, and then washing to remove cell surface bound protein. Values and error bars represent the mean and standard deviation of three independent experiments.

FIG. 12 provides a bar graph showing the efficiency of nano luciferase (nLuc) delivery to human prostate cancer cells (PC-3). PC-3 cells were treated with either nLuc or Ypep(Gly4Asn)-Ypep, then washed to remove cell surface bound protein. nLuc does not appreciably penetrate PC-3 cells; however, relatively high levels of internalized functional nLuc were observed in cells following treatment with Ypep(G4N)-nLuc.

FIG. 13 provides the amino acid sequences of Ypep and Ypep mutants.

### DETAILED DESCRIPTION OF THE INVENTION

The present invention is based, in part, on the identification of novel protein translocation domains (PTDs) capable of selectively delivering macromolecular biopolymers, including therapeutic agents and bioimaging agents, and nanoparticles to the interior of a target cell. PTDs, sometimes termed cell permeable proteins (CPPs) or membrane translocating sequences (MTSs) are small peptides that are able to ferry much larger molecules into cells independent of classical endocytosis. In addition, the present invention is also based, in part, on the development of a novel phage delivery system that utilizes PTDs and other targeting agents to effectively transduce target cells, e.g., for the delivery of macromolecular biopolymers, including therapeutic agents and bioimaging agents, and nanoparticles to the interior of the target cell, or to bind to a microorganism, such as a virus or bacterium, e.g., for delivery of an anti-viral or anti-bacterial agent to said virus of bacterium, respectively, or to bind a bioimaging agent to the virus or bacterium.

Macromolecular biopolymers, such as proteins, are of great value as research tools, therapeutics, and bioimaging reagents. For example, the size and complexity of proteins can often endow these reagents with the ability to potently and selectively recognize disease-relevant macromolecular surfaces that confound drug discovery efforts focused on traditional small molecules (<800 Da). In addition, a number of protein enzymes such as luciferase and horseradish peroxidase are used for bioimaging applications. In many ways, these reagents are ideally suited for imaging cells. For example, by virtue of their enzymatic activity, appreciable signal can be generated from a relatively small number of functional proteins.

However, perhaps the greatest barrier to the broader use of these reagents in medicine and basic research is the inability of most proteins to penetrate the lipid bilayer membrane of mammalian cells. In the context of therapy, this largely limits their use to receptors on the surface of a cell. However, proteins residing on the cell surface make up a small fraction of the proteome. In the context of bioimaging, internalization effectively locks the signal inside of cells containing the functional imaging enzyme. The potential utility of proteins with the ability to access the interior of mammalian cells provides a strong need to address barriers to the intracellular delivery of these agents in mammalian cells. As a result, the development of methods for functional protein delivery has received significant attention. Technologies including electroporation, microinjection, liposomes, lipid-linked compounds, nanoparticles, fusions to receptor ligands, arginine grafting, supercharged proteins, and protein transduction domains (PTDs) have been previously reported.

As described herein and demonstrated in the accompanying Example, the present invention includes novel PTDs capable of mediating transduction of target cells and delivery of macromolecular biopolymers, such as proteins, to the interior of the target cells, phage-based delivery systems that utilize PTDs and methods for the production of the same, as well as related methods of treating or preventing diseases, and performing bioimaging.

### DEFINITIONS AND ABBREVIATIONS

The words “a” and “an” denote one or more, unless specifically noted.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. In any embodiment discussed in the context of a numerical value used in conjunction with the term “about,” it is specifically contemplated that the term about can be omitted.

Unless the context requires otherwise, throughout the present specification and claims, the word “comprise” and variations thereof, such as, “comprises” and “comprising” are to be construed in an open, inclusive sense, that is as “including, but not limited to”.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

An “increased” or “enhanced” amount is typically a “statistically significant” amount, and may include an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 2.1, 2.2, 2.3, 2.4, etc.) an amount or level described herein.

A “decreased” or “reduced” or “lesser” amount is typically a “statistically significant” amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

A “composition” can comprise an active agent, e.g., a phage, and a carrier, inert or active, e.g., a pharmaceutically acceptable carrier, diluent or excipient. In particular embodiments, the compositions are sterile, substantially free of endotoxins or non-toxic to recipients at the dosage or concentration employed.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent or emulsifier which has been approved by the United States Food and Drug Administration as being acceptable for use in humans or domestic animals.

The terms “mammal” and “subject” include human and non-human mammals, such as, e.g., a mouse, rat, rabbit, monkey, cow, hog, sheep, goat, horse, swine, dog, or cat. “Mammal” includes humans and both domestic animals such as laboratory animals and household pets (e.g., cats, dogs, swine, cattle, sheep, goats, horses, and rabbits), and non-domestic animals such as wildlife and the like.

The terms “tissue” and “organ” are used according to their ordinary and plain meanings. Though tissue is composed of cells, it will be understood that the term “tissue” refers to an aggregate of similar cells forming a definite kind of structural material. Moreover, an organ is a particular type of tissue. In certain embodiments, the tissue or organ is “isolated,” meaning that it is not located within an organism.

By “fusion polypeptide” or “fusion protein” is meant a fusion polypeptide (protein) comprising first and second polypeptides encoded by first and second nucleic acid sequences, respectively, which are operatively linked (fused). As exemplified in certain embodiments herein, fusion proteins displayed on phage particles are fusions of an exoprotein, a protein not native to the phage proteome, and a phage coat protein.

By “exogenous polypeptide” or “exogenous protein” or “exoprotein” is meant a protein not normally encoded by the phage genome, but rather is foreign to the normal phage proteins. A typical exogenous polypeptide is any polypeptide of interest, including a PTD or PTD multimer peptide.

The term “surface of a phage or bacteriophage” or “surface of a phage particle” refers to the part of a phage or bacteriophage particle which is in contact with the medium in which the particle is contained. The surface of the phage particle is determined by the coat protein assembly (the assembled members of the protein coat of the particle).

By “phage coat protein” is meant those proteins forming the phage coat of naturally occurring bacteriophages. In filamentous bacteriophage, such as fl, fd, and M13, the coat proteins are gene III protein (pill or p3), gene VI protein (pVI or p6), gene VII protein (pVII or p7), gene VIII protein (pVIII or p8), and gene IX protein (pIX or p9). The sequences of the coat proteins of M13 as well as the differences between the closely related members of the filamentous bacteriophages are well known to one of ordinary skill in the art (see, e.g., Kay, B. K., Winter, J. & McCafferty, J., eds. (1996). Phage display of peptides and proteins: a laboratory manual. Academic Press, Inc., San Diego).

The term “buffer” as used herein denotes a pharmaceutically acceptable excipient, which stabilizes the pH of a pharmaceutical preparation. Suitable buffers are well known in the art. Suitable pharmaceutically acceptable buffers include but are not limited to acetate-buffers, histidine-buffers, citrate-buffers, succinate-buffers, tris-buffers and phosphate-buffers. In certain embodiments, the concentration of the buffer is from about 0.01 mM to about 1000 mM, about 0.1 mM to about 1000 mM, about 0.1 mM to about 500 mM, about 0.1 to about 200 mM, about 0.1 to about 100 mM, about 1 mM to about 1000 mM, about 1 mM to about 500 mM, about 1 mM to about 200 mM, about 1 mM to about 100 mM, about 1 mM to about 50 mM, about 2 mM to about 60 mM, about 4 mM to about 60 mM, or about 4 mM to about 40 mM, about 5 mM to about 20 mM, or about 5 mM to about 25 mM.

Pharmaceutically acceptable “cryoprotectants” are known in the art and include without limitation, e.g., sucrose, trehalose, and glycerol. Pharmaceutically acceptable cryoprotectants provide stability protection of compositions, or one or more active ingredients therein, from the effects of freezing and/or lyophilization.

The term “stabilizer” indicates a pharmaceutical acceptable excipient, which protects the active pharmaceutical ingredient(s) or agents(s) and/or the composition from chemical and/or physical degradation during manufacturing, storage and application. Stabilizers include, but are not limited to, sugars, amino acids, polyols, surfactants, antioxidants, preservatives, cyclodextrines, e.g. hydroxypropyl-β-cyclodextrine, sulfobutylethyl-β-cyclodextrin, β-cyclodextrin, polyethyleneglycols, e.g. PEG 3000, PEG 3350, PEG 4000, PEG 6000, albumin, e.g. human serum albumin (HSA), bovine serum albumin (BSA), salts, e.g. sodium chloride, magnesium chloride, calcium chloride, and chelators, e.g. EDTA. Stabilizers may be present in the composition in an amount of about 0.1 mM to about 1000 mM, about 1 mM to about 500 mM, about 10 to about 300 mM, or about 100 mM to about 300 mM.

A “lyoprotectant” refers to a pharmaceutically acceptable substance that stabilizes a protein, nucleic acid or other active pharmaceutical ingredient(s) or agent(s) during lyophilization. Examples of lyoprotectants include, without limitation, sucrose, trehalose or mannitol.

A “preservative” is a natural or synthetic chemical that is added to products such as foods, pharmaceutical compositions, paints, biological samples, wood, etc. to prevent decomposition by microbial growth or by undesirable chemical changes. Preservative additives can be used alone or in conjunction with other methods of preservation. Preservatives may be antimicrobial preservatives, which inhibit the growth of bacteria and fungi, or antioxidants such as oxygen absorbers, which inhibit the oxidation of constituents. Examples of antimicrobial preservatives include benzalkonium chloride, benzoic acid, cholorohexidine, glycerin, phenol, potassium sorbate, thimerosal, sulfites (sulfur dioxide, sodium bisulfite, potassium hydrogen sulfite, etc.) and disodium EDTA. Other preservatives include those commonly used in patenteral protein compositions such as benzyl alcohol, phenol, m-cresol, chlorobutanol or methylparaben.

As used in the specification and appended claims, unless specified to the contrary, the following terms have the meaning indicated:

“Optional” or “optionally” means that the subsequently described event of circumstances may or may not occur, and that the description includes instances where said event or circumstance occurs and instances in which it does not.

“Pharmaceutical composition” refers to a formulation of a compound and a medium generally accepted in the art for the delivery of the biologically active compound to mammals, e.g., humans. Such a medium may include any pharmaceutically acceptable carriers, diluents or excipients therefore.

“Therapeutically effective amount” refers to that amount of a compound or composition of the invention, e.g., a genetically modified bacteriphage, that, when administered to a mammal, e.g., a human, is sufficient to effect treatment, as defined below, of a disease or condition in the mammal, e.g., a human. The amount of a compound or composition of the invention that constitutes a “therapeutically effective amount” will vary depending on the compound or composition, the condition and its severity, the manner of administration, and the age of the mammal to be treated, but can be determined routinely by one of ordinary skill in the art having regard to his own knowledge and to this disclosure.

“Treating” or “treatment” as used herein covers the treatment of the disease or condition of interest, e.g., tissue injury, in a subject or mammal, e.g., a human, having the disease or condition of interest, and includes: (i) preventing or inhibiting the disease or condition from occurring in a mammal, in particular, when such subject or mammal is predisposed to the condition but has not yet been diagnosed as having it; (ii) inhibiting the disease or condition, i.e., arresting or slowing its development; (iii) relieving the disease or condition, i.e., causing regression of the disease or condition; or (iv) relieving the symptoms resulting from the disease or condition. As used herein, the terms “disease,” “disorder,” and “condition” may be used interchangeably or may be different in that the particular malady or condition may not have a known causative agent (so that etiology has not yet been worked out), and it is, therefore, not yet recognized as a disease but only as an undesirable condition or syndrome, wherein a more or less specific set of symptoms have been identified by clinicians.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, He, Phe, Tyr, Trp, Lys, Arg, His, Asp, Glu, Asn, Gin, Cys and Met) occurs in both sequences to yield the number of matched positions, dividing the number of matched positions by the total number of positions in the window of comparison (i.e., the window size), and multiplying the result by 100 to yield the percentage of sequence identity.

Terms used to describe sequence relationships between two or more polynucleotides or polypeptides include “reference sequence,” “comparison window,” “sequence identity,” “percentage of sequence identity” and “substantial identity.” A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al, 1997, Nucl. Acids Res. 25:3389. A detailed discussion of sequence analysis can be found in Unit 19.3 of Ausubel et al, “Current Protocols in Molecular Biology,” John Wiley & Sons Inc, 1994-1998, Chapter 15.

Calculations of sequence similarity or sequence identity between sequences (the terms are used interchangeably herein) are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In certain embodiments, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch, (1970, J. Mol. Biol. 48: 444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frame shift gap penalty of 5.

The percent identity between two amino acid or nucleotide sequences can be determined using the algorithm of E. Meyers and W. Miller (1989, Cabios, 4: 11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases, for example, to identify other family members or related sequences. Such searches can be performed using the NBLAST and XBLAST programs (version 2.0) of Altschul, et al, (1990, J. Mol. Biol, 215: 403-10). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997, Nucleic Acids Res, 25: 3389-3402). When utilizing BLAST and Gapped BLAST programs, the default parameters of the respective programs (e.g., XBLAST and NBLAST) can be used.

A PTD or targeting agent is said to “specifically bind” a target cell or microorganism, if it binds at a detectable level (within, for example, an ELISA assay) with the target cell or microorganism, and does not react detectably in a statistically significant manner with unrelated cells or microorganisms under similar conditions. In certain embodiments, a PTD or targeting agent is said to “preferentially bind” a target cell or microorganism, if it binds with an at least two-fold, at least five-fold, at least 10-fold, at least 50-fold, at least 100-fold greater affinity to the target cell or microorganism as compared to an unrelated cell or microorganism, respectively, such as, e.g., a non-cancer cell from the same or a different tissue or cell type, or a cell from a different tissue. In particular embodiments wherein the target cell is a disease cell, e., a tumor cell, an unrelated cell is a non-diseased cell of the same tissue type. The strength or affinity of binding can be expressed in terms of the dissociation constant (Ka) of the interaction, wherein a smaller Ka represents a greater affinity. Binding properties of peptides and other binding agents can be quantified using methods well known in the art. In certain illustrative embodiments, a targeting peptide or targeting agent has an affinity for a target cell or target microorganism of at least about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM. In certain embodiments, the affinity of the targeting peptide or targeting agent for the target cell or target microorganism is stronger than its affinity for an unrelated cell or microorganisms, respectively, typically by about 1.5×, 2×, 2.5×, 3×, 3.5×, 4×, 4.5×, 5×, 6×, 7×, 8×, 9×, 10×, 15×, 20×, 25×, 30×, 40×, 50×, 60×, 70×, 80×, 90×, 100×, 200×, 300×, 400×, 500×, 600×, 700×, 800×, 900×, 1000× or more (including all integers in between). In certain embodiments, a targeting peptide or targeting agent has an affinity for a target cell or target microorganism of at least about 0.05, 0.1, 0.25, 0.5, 0.75, 1, 2, 3, 4, 5, 6, 7, 8, 9, 10, 11, 12, 13, 14, 15, 16, 17, 18, 19, or 20 μM.

As used herein, an “antibody” includes whole antibodies and any antigen binding fragment or a single chain thereof. Thus the term “antibody” includes any protein or peptide containing molecule that comprises at least a portion of an immunoglobulin molecule having biological activity of binding to the antigen. Examples of such may comprise a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein. Antibodies include monoclonal antibodies and polyclonal antibodies.

The term “antibody fragment” includes a portion of an antibody that is an antigen binding fragment or single chains thereof. An antibody fragment can be a synthetically or genetically engineered polypeptide. Examples of binding fragments encompassed within the term “antigen-binding portion” of an antibody include: (i) a Fab fragment, a monovalent fragment consisting of the V_{L}, V_{H}, C_{L} and C_{H1} domains, (ii) a F(a′)₂ fragment, a bivalent fragment comprising two Fab fragments linked by a disulfide bridge at the hinge region, (iii) a Fd fragment consisting of the V_{H} and C_{H1} domains, (iv) a Fv fragment consisting of the V_{L} and V_{H} domains of a single arm of an antibody, (v) a dAb fragment (Ward et al, (1989) Nature 341 544-546), which consists of a V_{H} domain, and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv), see e.g., Bird et al. (1988) Science 242 423-426, and Huston et al. (1988) Proc Natl Acad Sci USA 85 5879-5883). Such single chain antibodies are also intended to be encompassed within the term “antigen-binding fragment” of an antibody. These antibody fragments are obtained using conventional techniques known to those in the art, and the fragments can be screened for utility in the same manner as whole antibodies.

The term “aptamer” refers to a peptide or nucleic acid that has an inhibitory effect on a target. Inhibition of the target by the aptamer can occur by binding of the target, by catalytically altering the target, by reacting with the target in a way which modifies the target or the functional activity of the target, by ionically or covalently attaching to the target as in a suicide inhibitor or by facilitating the reaction between the target and another molecule. Aptamers can be peptides, ribonucleotides, deoxyribonucleotides, other nucleic acids or a mixture of the different types of nucleic acids. Aptamers can comprise one or more modified amino acid, bases, sugars, polyethylene glycol spacers or phosphate backbone units as described in further detail herein. Aptamers can be pegylated or unpegylated. For example, one or more polyethylene glycol chains can be linked to the 5′end of a nucleic acid aptamer via a linker.

### Protein Transduction Domains (PTDs) and Related Fusion Proteins

In many ways, PTDs are ideally suited as protein delivery reagents. The proteinogenic amino acid composition and relatively small size (typically 7-20 L-amino acids) of these reagents allow researchers to express protein-PTD fusions with relative ease. A number of PTDs such as HIV-1 transactivator of transcription (Tat) peptide, the Drosophila Antennapedia-derived penetratin peptide, and polyarginine have been used extensively for protein delivery. However, relatively low uptake efficiency and lack of selectivity for diseased cells over healthy cells limits the full potential of these reagents. The development of new PTDs capable of potently and selectively delivering functional proteins, such as enzymes, to diseased cells would potentially expand the biomedical utility of protein-based approaches to medicine.

In one aspect, the present invention provides novel PTDs, including PTDs that selectively bind to and/or mediate transduction of a target cell. In particular embodiments, the target cell is a tumor cell, e.g., a prostate tumor cell. These PTDs may be used, e.g., to direct the binding and internalization of various therapeutic agents or imaging agents to target cells.

As demonstrated in the accompanying Examples, the PTD referred to as Ypep, and related mutant Ypep peptides and Ypep multimer peptides, selectively targeted Green Fluorescent Protein (GFP) and M13 bacteriophage to the interior of PC-3 cells, a human prostate cancer cell line with high-metastatic potential. PC-3 cells are one of the most commonly used human prostate cancer cell lines in basic and translational research, and are useful in investigating the biochemical changes in advanced prostate cancer cells and in assessing their response to chemotherapeutic agents. Moreover, they can be used to create subcutaneous tumors in mice in order to investigate response to a therapeutic in the context of a larger organism. Reagents that selectively recognize PC-3 cells are interesting, since these cells do not express appreciable levels of prostate-specific membrane-bound antigen (PSMA), which is the most commonly used marker for prostate cancer cell detection and targeted drug delivery. Therefore, PC-3 cells, or prostate cancer cells with a similar phenotype, would likely evade targeted imaging or drug delivery strategies centered on PSMA recognition.

As described herein, GFP or bacteriophage bearing multiple copies of Ypep potently and selectively penetrated PC-3 cells. In addition, numerous Ypep mutants showed significantly increased uptake efficiency and prostate cancer cell selectivity as compared to Ypep. These studies establish the functional utility of Ypep-dependent delivery of exogenous proteins to the interior of PC-3-like prostate cancer cells and establish that Ypep and Ypep mutants are well suited to serve as reagents for PC-3-like cell-selective delivery of imaging agents and macromolecular biopolymers, such as enzymatic proteins, for basic research and therapeutic applications.

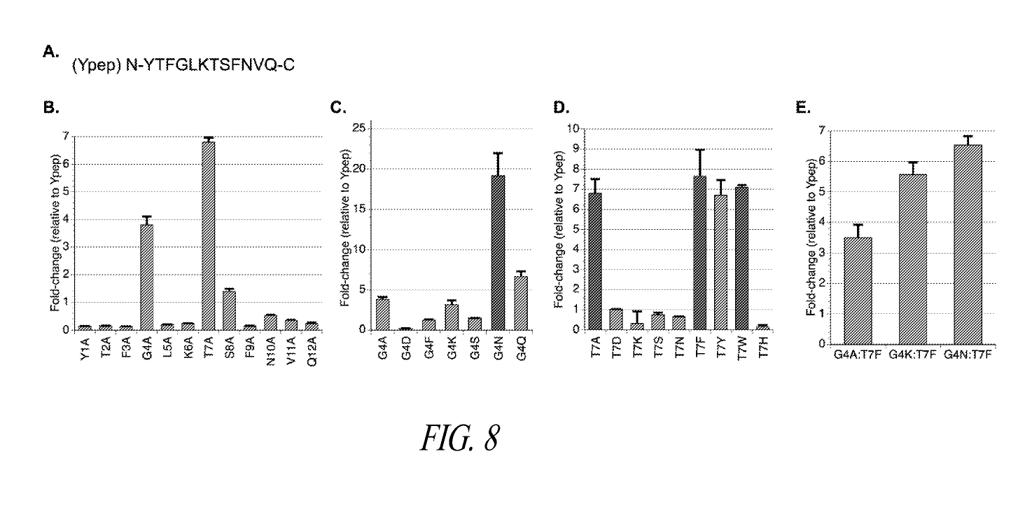

In one embodiment, a PTD of the invention is a peptide referred to herein as Ypep, which has the amino acid sequence shown in FIG. 8. In related embodiments, other PTDs of the invention have at least 80%, at least 90%, at least 95%, at least 98%, or at least 99% amino acid sequence identity with the amino acid sequence of Ypep. In further related embodiments, PTDs of the invention are peptides that comprise the Ypep amino acid sequence, or they comprise a region having at least 80%, at least 90%, at least 95%, at least 98%, or at least 99% amino acid sequence identity with the amino acid sequence of Ypep.

In particular embodiments, PTDs of the invention include peptides that comprise one, two, three, four, at least one, at least two, at least three, at least four, one to four, one to three, or one to two amino acid substitutions as compared to the amino acid sequence of Ypep. For example, PTDs of the invention include peptides comprising or consisting of any of the following single mutations or alterations as compared to the Ypep amino acid sequence: Gly4Ala, Thr7Ala, Ser8Ala, Gly4Lys, Gly4Asn, Gly4Gln, Thr7Ala, Thr7Phe, Thr7Tyr, or Thr7Trp; or any of the following double mutations as compared to the Ypep amino acid sequence:Gly4Ala:Thr7Phe, Gly4Lys:Thr7Phe, or Gly4Asn:Thr7Phe. PTDs of the invention also include peptides comprising or consisting of any of the mutant forms of Ypep shown in FIG. 8.

In certain embodiments, the invention includes peptides comprising or consisting of two, three, four, five, two or more, three or more, four or more, or five or more PTDs described herein, which may be referred to herein as PTD multimer peptides. Each of the PTDs present in the PTD multimer peptide may be the same, or one or more may be different. Each of the PTDs present in the PTD multimer peptide may be immediately adjacent to one other, or there may be one or more additional amino acids present between one or more of the PTDs present in the PTD multimer peptide. In certain embodiments, the amino acids present between one or more PTDs present in the PTD multimer peptide comprise a linker sequence. Examples of illustrative linkers that can be used include (GGS)₃ and (GGS)₄ linkers, where G is glycine and S is serine.

In related embodiments, the invention includes fusion proteins comprising one or more PTDs or PTD multimer peptides described herein fused to another amino acid sequence. In certain embodiments of fusion proteins comprising a PTD of the invention, the PTD(s) or PTD multimer peptide is immediately adjacent to the other protein, while in other embodiments, one or more additional amino acids are present between the PTD(s) or PTD multimer peptide and the other protein.

In certain embodiments, the other amino acid sequence of the fusion protein is a bacteriophage coat protein or portion thereof. In particular embodiments, the bacteriophage coat protein is a p3, p6, p7, p8, or p9 coat protein. In certain embodiments, the bacteriophage coat protein is an M13 bacteriophage coat protein. In particular embodiments, the phage coat protein is M13 p3, M13 p6, M13 p7, M13 p8, or M13 p9. In certain embodiments, the fusion protein comprises one or more PTDs or PTD multimer peptides described herein fused to an M13 bacteriophage p3 coat protein or fragment thereof.

M13 is a filamentous bacteriophage composed of circular single stranded DNA (ssDNA), which is 6407 nucleotides long, encapsulated in approximately 2700 copies of the major coat protein P8, and capped with 5 copies of two different minor coat proteins (P9, P6, P3) on the ends. The phage coat is primarily assembled from a 50 amino acid protein called pVIII (or p8), which is encoded by gene VIII (or g8) in the phage genome. For a wild type M13 particle, it takes approximately 2700 copies of p8 to make the coat about 900 nm long. The coat's dimensions are flexible though and the number of p8 copies adjusts to accommodate the size of the single stranded genome it packages. For example, when the phage genome was mutated to reduce its number of DNA bases (from 6.4 kb to 221 bp), then the number of p8 copies was decreased to fewer than 100, causing the p8 coat to shrink in order to fit the reduced genome. The phage appear to be limited at approximately twice the natural DNA content. However, deletion of a phage protein (p3) prevents full escape from the host E. coli, and phage that are 10-20× the normal length with several copies of the phage genome can be seen shedding from the E. coli host.

There are four other proteins on the phage surface, two of which have been extensively studied. At one end of the filament are five copies of the surface exposed pIX (p9) and a more buried companion protein, pVII (pi). If p8 forms the shaft of the phage, p9 and p7 form the “blunt” end that is seen in the micrographs. These proteins are very small, containing only 33 and 32 amino acids respectively, though some additional residues can be added to the N-terminal portion of each which are then presented on the outside of the coat. At the other end of the phage particle are five copies of the surface exposed pill (p3) and its less exposed accessory protein, pVI (p6). These form the rounded tip of the phage and are the first proteins to interact with the E. coli host during infection. p3 is also the last point of contact with the host as new phage bud from the bacterial surface.

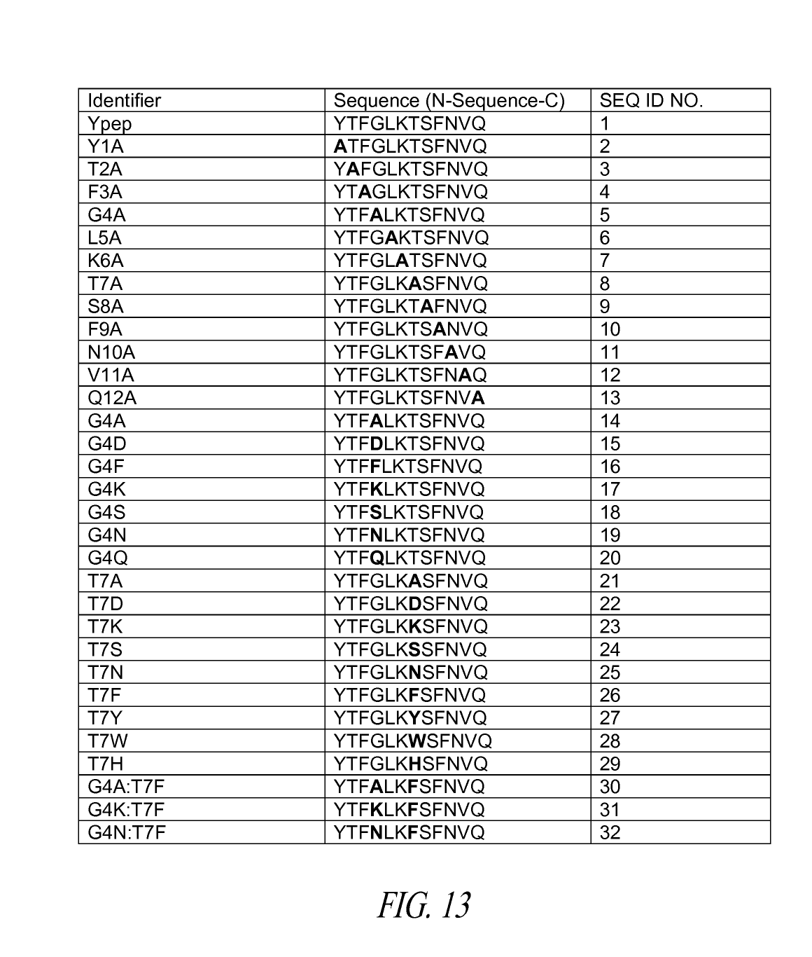

In a related embodiment, the invention includes a polynucleotide comprising or consisting of a sequence that encodes a PTD, a PTD multimer peptide, or a fusion protein of the invention. It is understood that due to the degeneracy of the genetic code, a variety of different polynucleotide sequences may encode PTDs, PTD multimer peptides, or fusion proteins described herein, and all such polynucleotides are encompassed by the invention. In particular embodiments, the encoded PTD is Ypep; any of the following single mutations or alterations as compared to the Ypep amino acid sequence: Gly4Ala, Thr7Ala, Ser8Ala, Gly4Lys, Gly4Asn, Gly4Gln, Thr7Ala, Thr7Phe, Thr7Tyr, or Thr7Trp; or any of the following double mutations as compared to the Ypep amino acid sequence:Gly4Ala:Thr7Phe, Gly4Lys:Thr7Phe, or Gly4Asn:Thr7Phe. The amino acid sequences of these Ypep mutants are provided in FIG. 13.

In a further related embodiment, the invention includes a vector comprising a polynucleotide of the invention, i.e., a polynucleotide comprising or consisting of a sequence that encodes a PTD, a PTD multimer peptide, or a fusion protein of the invention. The vector may be a replicating or a non-replicating vector. In certain embodiments, the vector is a plasmid vector, while in other embodiments, the vector is a viral vector. In certain embodiments, the vector is a cloning vector, and in certain embodiments, the vector is an expression vector. In particular embodiments, the vector may include one or more additional polynucleotide sequences, such as those necessary for replication or expression of an encoded protein, such as an origin of replication, a promoter a polyA sequence. In certain embodiments, such vectors may be used for expression of an encoded protein in bacterial or mammalian cells. In other embodiments, such vectors may be used for the production of a virus or bacteriophage comprising a PTD of the invention.

In yet another related embodiment, the invention includes a virus or bacteriophage comprising a PTD, a PTD multimer peptide, or a fusion protein of the invention, or a virus or bacteriophage comprising a nucleic acid encoding a PTD, a PTD multimer peptide, or a fusion protein of the invention. In certain embodiments, the bacteriophage is an M13 bacteriophage. In particular embodiments, the M13 bacteriophage comprises a fusion protein comprising a PTD and a bacteriophage coat protein, e.g., the M13 p3 coat protein.

Methods for producing PTDs and related peptides, fusion proteins, polynucleotides and vector are known in the art. For example, PTDs and related peptides may be produced synthetically or recombinantly, and related fusion proteins, polynucleotides and vectors may be produced recombinantly using routine molecular and cellular biology methodologies.

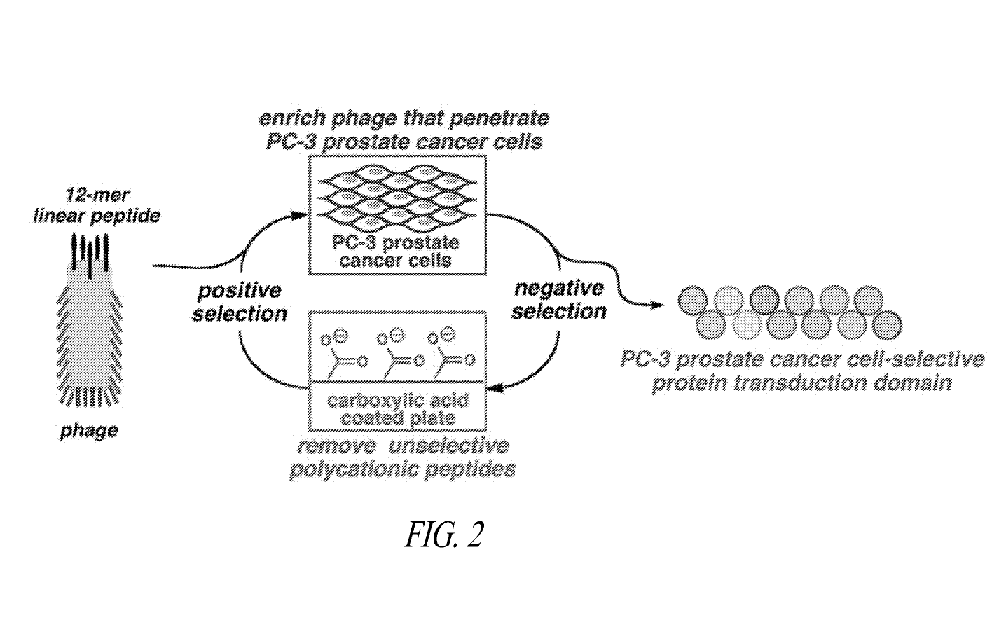

The invention further provides methods for identifying and producing target-cell specific PTDs, comprising selecting for a PTD that mediates transduction of a target cell, as described in Example 1. Using a scheme similar to that shown in FIG. 2, phage display evolution is used to generate cancer cell-selective penetrating phage. This approach can be used to identify phage that selectively penetrate any cancer cell—it is simply a matter of which cancer cell is used in the positive selection. In particular embodiments, the method comprises screening phage, e.g., M13 phage, by one, two, three, one or more, two or more, or three or more rounds of selection, each of which may comprise: (1) contacting target cells with a plurality of phage that express on their surface a fusion protein of a phage coat protein and a peptide comprising or consisting of a candidate PTD; (2) enriching for phage that penetrate the target cells; and (3) depleting phage that non-specifically bind the target cells, e.g., by incubating the penetrating phage with a polyanionic tissue culture plate (e.g., coated with carboxylic acid), thereby enriching for phage that comprise a PTD for the target cell. After one or more rounds of selection, a polynucleotide encoding a portion of the fusion protein comprising the candidate PTD may be sequenced, thus identifying the target cell-specific PTD. In certain embodiments, the plurality of phage constitute a phage display library, wherein different phage within the library comprise fusion proteins comprising different candidate PTDs. In particular embodiments, the phage coat protein of the fusion protein is p3, p6, p7, p8, or p9. In one embodiment, it is p3. In particular embodiments, the target cell is a disease cell, e.g., a tumor cell. In particular embodiments, the target cell is a specific cell type, e.g., any diseased cell including, but not limited to, a cancer cell at any stage of growth, virus, an adipose cell, or cancer stem cell. In particular embodiments, a target cell is a microorganism, e.g., a bacterium. One of skill will also appreciate that aspects of the present invention may be modified for delivery of a macromolecular biopolymer to a target microorganism, such as, e.g., a bacterium or a virus.

### Genetically Modified Bacteriophage

In another aspect, the invention includes a genetically modified bacteriophage useful in delivering a macromolecular biopolymer, such as a protein therapeutic agent or an imaging agent, to the interior of a target cell, or to bind or associate a macromolecular biopolymer, such as a protein antibiotic or antiviral agent or an imaging agent, to a target microorganism, such as a bacteria or a virus. A macromolecule is a large molecule commonly created by polymerization of smaller subunits. In certain embodiments, macromolecular biopolymers include nucleic acids (e.g., single-stranded, double-stranded or triple-stranded polynucleotides, DNA, RNA, mRNA or cDNA), all types of proteinaceous reagents (e.g., proteins (e.g., both natural and synthetic), peptides, antibodies, antibody fragments, or nanobodies), and carbohydrates.

The genetically modified bacteriophage, also referred to as genetically modified or engineered multifunctional phage, of the invention are optimal vehicles for macromolecular therapeutic delivery or nanoparticle magnetic resonance imaging (MRI) imaging reagent delivery in vitro and in vivo, such as, e.g., iron oxide nanoparticle-binding prostate cancer cell-penetrating phage as reagents for imaging prostate cancer cells. These genetically engineered multifunctional phage are a macromolecular delivery vehicle that is shelf stable, inexpensive to prepare and store, and robust. Accordingly, they have the real potential to significantly expand the use of macromolecular therapeutics in the healthcare marketplace. In addition, the genetically engineered multifunctional phage are inexpensive, robust, and simple reagents that can be used for imaging tumors. Unlike antibodies and other protein therapeutics, phage can be freeze-dried and compressed into a pill without altering their function, simplifying their transport and storage. Phage are stable at temperatures as high as 55° C., and typically have shelf lives up to 14 months. The preparation, isolation, and purification of phage are simple and inexpensive, decreasing the cost associated with their mass production and shipment.

Genetically modified phage of the invention may be derived from any type of bacteriophage. A “bacteriophage” (informally, phage) is a virus that normally infects and replicates within bacteria. Bacteriophages are composed of proteins that encapsulate a DNA or RNA genome, and may have relatively simple or elaborate structures. Their genomes may encode as few as four genes, and as many as hundreds of genes. Bacteriophages may have a lytic cycle or a lysogenic cycle, and a few viruses are capable of carrying out both. With lytic phages such as the T4 phage, bacterial cells are broken open (lysed) and destroyed after immediate replication of the virion. In contrast, the lysogenic cycle does not result in immediate lysing of the host cell. Those phages able to undergo lysogeny are known as temperate phages. Their viral genome will integrate with host DNA and replicate along with it fairly harmlessly, or may even become established as a plasmid. The virus remains dormant until host conditions deteriorate, perhaps due to depletion of nutrients; then, the endogenous phages (known as prophages) become active. At this point, they initiate the reproductive cycle, resulting in lysis of the host cell. As the lysogenic cycle allows the host cell to continue to survive and reproduce, the virus is reproduced in all of the cell's offspring. An example of a bacteriophage known to follow the lysogenic cycle and the lytic cycle is the phage lambda of E. coli. In certain embodiments, the genetically modified phage is derived from a lytic phage, whereas in other embodiments, it is derived from a lysogenic phage, or a phage that is both lytic and lysogenic.

The general stages to a viral life cycle are: infection, replication of the viral genome, assembly of new viral particles and then release of the progeny particles from the host. Filamentous phage use a bacterial structure known as the F pilus to infect E. coli, with the M13 p3 tip contacting the TolA protein on the bacterial pilus. The phage genome is then transferred to the cytoplasm of the bacterial cell where resident proteins convert the single stranded DNA genome to a double stranded replicative form (“RF”). This DNA then serves as a template for expression of the phage genes.

To enter a host cell, bacteriophages attach to specific receptors on the surface of bacteria, including lipopolysaccharides, teichoic acids, proteins, or even flagella. This specificity means a bacteriophage can infect only certain bacteria bearing receptors to which they can bind. By introducing a PTD to a phage coat protein, modified phage of the invention are capable of binding to and entering cells other than bacterial cells, including mammalian cells, such as human cells.

In certain embodiments, the modified bacteriophage is derived from one of the following: Lambda phage (λphage), T2 phage, T4 phage, T7 phage, T12 phage, R17 phage, M13 phage, MS2 phage, G4 phage, PI phage, Enterobacteria phage P2, P4 phage, Phi X 174 phage, N4 phage, Pseudomonas phage Φ6, Φ29 phage, or 186 phage. In addition, other viruses could be used as a genetically-encodable template for multifunctional display and/or conjugation of cell-targeting and therapeutic and/or imaging reagents.

Lytic phage has been used as an antibacterial reagent in humans in Eastern Europe and France for over ninety years. These studies and others have shown that phage is generally non-toxic to mammals, and does not cause an appreciable immune response. In certain embodiments, the genetically modified phage is a filamentous bacteriophage, e.g., M13 phage. M13 is composed of circular single stranded DNA (ssDNA) which is 6407 nucleotides long encapsulated in approximately 2700 copies of the major coat protein P8, and capped with 5 copies of two different minor coat proteins (P9, P6, P3) on the ends. The minor coat protein P3 attaches to the receptor at the tip of the F pilus of the host Escherichia coli. It is a non-lytic virus. M13 phage have fast extravasation (on the order of minutes) and are cleared relatively slowly from circulation (circulation half-life of 4.5 hours in C57BL6 mice). Finally, M13 phage has excellent tissue biodistribution in mice. Taken together, this suggests that mammals, including humans, can be treated with phage without experiencing a severe immune response, those phage are not quickly secreted, or sequestered by the liver or other organs, and thus, tumor cell-selective cell-penetrating phage will thus have the opportunity to identify their tumor target when administered to an organism.

Phage can be genetically modified to display functional peptides or proteins on five distinct phage coat proteins (p3, p6, p7, p8, and p9). Using basic techniques and methods, phage may be genetically engineered such that they are transformed into optimized targeted delivery vehicles suitable for their intended purpose, e.g., delivery of therapeutic macromolecules or delivery of imaging reagents to a target cell or microorganism.

In certain embodiments, the invention provides a genetically modified bacteriophage comprising: a targeting agent (e.g., a peptide) that mediates transduction of a target cell or target microorganism (e.g., a peptide comprising or consisting of a PTD), wherein the targeting agent is displayed on the phage surface; and one or more conjugation peptide displayed on the phage surface. In certain embodiments, the targeting agent specifically binds a target cell or microorganism of interest or preferentially binds a target cell or microorganism of interest as compared to other cell types or microorganisms, respectively.

In various embodiments, the targeting agent is a peptide (e.g., a cyclic peptide), a peptide mimetic, a protein, an antibody or fragment thereof, an aptamer, a nanobody, or a nucleic acid, such, e.g., a single-stranded, double-stranded or triple-stranded RNA or DNA, or a hybrid DNA and RNA molecule.

In particular embodiments, the targeting peptide comprises or consists of a PTD, or a PTD multimer peptide. In particular embodiments, the targeting peptide comprises or consists of any of the PTDs or PTD multimer peptides described herein, such as, e.g., Ypep or a variant or mutant thereof. In certain embodiments, the targeting peptide is fused to a coat protein, such as p3, p6, p7, p8, or p9. In particular embodiments, it is fused to p3. In particular embodiments, the targeting peptide binds to a disease cell e.g., a tumor cell, such as a prostate tumor cell. However, it is appreciated that any particular cell, e.g., any type of cancer cell, can be targeted, particularly given the number and diversity of targeting reagents.

In certain embodiments, a conjugation peptide is fused to a coat protein in order to attach a macromolecular biopolymer or nanoparticle, e.g., a therapeutic agent, antimicrobial agent, or an imaging reagent, to the phage, e.g., either covalently or non-covalently. In particular embodiments, the conjugation peptide is a linker peptide, e.g., a peptide that can be used to covalently attach the fused coat protein to a macromolecular biopolymer, such as a protein (e.g., an antibody or fragment thereof). In certain embodiments, the conjugation peptide is a binding peptide, e.g., a peptide capable of binding an imaging reagent. In certain embodiments, the conjugation peptide is cleavable, e.g., such that the macromolecular biopolymer may be cleaved from the coat protein. In one embodiment, the conjugation peptide is a protease-cleavable linker. One example of a protease-cleavable linker is a peptide comprising the cathepsin B cleavage site. In one embodiment, a cathepsin B cleavage site is ALAL (i.e., alanine, leucine, alanine, leucine). In other embodiments, the conjugation peptide is non-cleavable, e.g., such that the imaging reagent is maintained bound to the phage within the cell. In certain embodiments, the conjugation peptide is fused to a coat protein, such as p3, p6, p7, p8, or p9. In particular embodiments, it is fused to p8.

A variety of peptide linkers and binding peptides that may be used as conjugation peptides to conjugate a macromolecular biopolymer, e.g., a protein, peptide or polynucleotide, to a phage coat protein are known in the art. In certain embodiments, the conjugation peptide is a (GGS)₃ or (GGS)₄ linker; however, all possible linkers within the proteinogenic and chemical space may be used. In addition, synthetic linkers involving organic matter may be used for chemical conjugation.

In certain embodiments, a conjugation peptide comprises or consists of one or more sortase recognition domain or sortase bridging domain, as described, e.g., in Proft, T. Biotechnol Lett (2010) 32:1-10. Sortases are transpeptidases produced by Gram-positive bacteria to anchor cell surface proteins covalently to the cell wall. The Staphylococcus aureus sortase A (SrtA) cleaves a short C-terminal recognition motif (LPXTG) (referred to herein as a sortase recognition domain) on the target protein followed by the formation of an amide bond with the pentaglycine cross-bridge in the cell wall (this pentaglycine sequence is referred to herein as a sortase bridging domain). This specific reaction has been exploited for a range of biotechnology applications, including the incorporation of non-native peptides and non-peptidic molecules into proteins, the generation of nucleic acid-peptide conjugates and neoglycoconjugates, protein circularization, and labeling cell surface proteins on living cells, as well as conjugation of a protein to M13 bacteriophage.

In various embodiments, the sortase recognition domain is a sortase A recognition domain or a sortase B recognition domain. In particular embodiments, the sortase recognition domain comprises or consists of the amino acid sequence: LPTGAA, LPTGGG, LPKTGG, LPETG, LPXTG or LPXTG(X)n, where X is any amino acid, and n is 0, 1, 2, 3, 4, 5, 7, 8, 9, 10, in the range of 0-5 or 0-10, or any integer up to 100. In particular embodiments, the conjugation peptide is a sortase recognition domain, which is fused to a coat protein, e.g., p8. The conjugation peptide, e.g., a sortase recognition domain, could, however, be fused to any coat protein. In addition, the sortase recognition domain could also be fused to any coat protein on phage.

In particular embodiments, the sortase bridging domain comprises one or more glycine residues at one of its termini. In certain embodiments, the one or more glycine residues may optionally be: Gly, (Gly)₂, (Gly)₃, (Gly)₄, or (Gly)₅. In particular embodiments, the conjugation peptide is a sortase bridging domain, which is fused to a coat protein, e.g., p8. The conjugation peptide, e.g., a sortase bridging domain, could, however, be fused to any coat protein. In addition, the sortase bridging domain could also be fused to any coat protein on phage.

In certain embodiments, the macromolecular biopolymer being coupled to the phage coat protein comprises a sortase bridging domain or sortase recognition domain capable of being fused to the sortase recognition domain or sortase bridging domain, respectively, present on the coat protein.

In other embodiments, the conjugation peptide is capable of binding directly to a macromolecular biopolymer, therapeutic agent or imaging reagent (e.g., nanoparticle). For example, in one embodiment, the conjugation peptide is an iron oxide nanoparticle-binding peptide, such as, e.g., a triglutamate motif, e.g., GGG.

In certain embodiments, bacteriophage of the invention are engineered to comprise a first conjugation peptide fused to a first coat protein, and a second conjugation peptide fused to a second coat protein. In various embodiments, the same or different macromolecular polymers and/or imaging agents may be coupled to the first and second coat proteins via the first and second conjugation peptides, respectively. In particular embodiments, the first phage coat protein is p8 and the second phage coat protein is p9.

In particular embodiments, the genetically modified bacteriophage comprises one or more macromolecular biopolymer, e.g., a polypeptide, peptide or polynucleotide, or imaging agent fused to or bound to the one or more conjugation peptide.

Without wishing to be bound by theory, it is thought that one mechanism of phage uptake is via an endocytic process. Therefore, delivery of a functional macromolecular biopolymer, e.g., a therapeutic agent, may require release from the endosome to the cytosol. Various endosomolytic peptides have been reported, which disrupt endosome structure, resulting in the release of cargo (including protein cargo). Therefore, in particular embodiments, the genetically modified phage further comprises an endosomolytic peptide fused to a phage coat protein, in order to achieve functional delivery of the macromolecular polymer to the cytosol of the cell, e.g., delivery of a therapeutic agent to the cytosol of a diseased cell. In particular embodiment, the endosomolytic peptide is fused to a phage coat protein selected from p3, p6, p7, p8 or p9. In one embodiment, it is fused to p9.

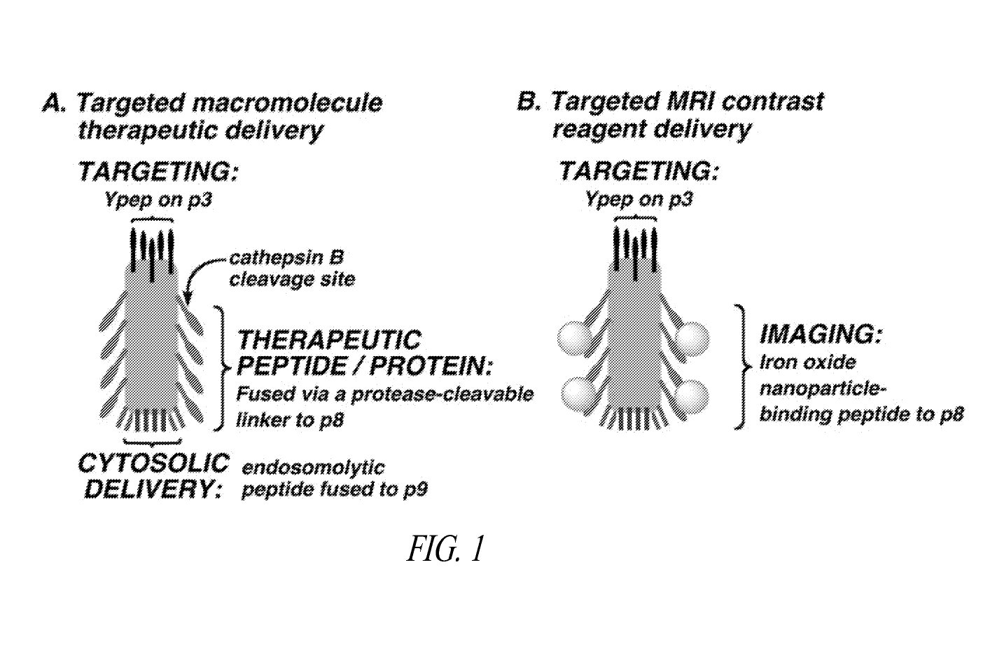

Accordingly, in particular embodiments, a genetically modified phage of the invention comprises: (1) a targeting agent (e.g., a targeting peptide) fused to p3, which is displayed on the phage surface and binds to and/or mediates transduction of a target cell or microorganism; (2) a conjugation peptide fused to p8, which is displayed on the phage surface and is capable of fusing with or binding to a macromolecular biopolymer (such as, e.g., a protein (e.g., an antibody), peptide, or polynucleotide); and, optionally, (3) an endosomolytic peptide fused to p9, which is displayed on the phage surface. A schematic diagram of certain embodiments, of illustrative genetically modified phage of the invention is provided in FIG. 1. In particular embodiments, the conjugation peptide is bound to the macromolecular biopolymer, e.g., via a coupling peptide fused to or associated with the macromolecular biopolymer.

Modified phage of the invention may be produced without a conjugated or bound macromolecular biopolymer (e.g., a therapeutic agent or an imaging reagent), or with a macromolecular biopolymer conjugated (e.g., fused) or bound to the modified phage. In certain instances, it is desirable to produce a modified phage not yet having any bound biomolecular agent, such that an appropriate or desired macromolecular biopolymer can be bound prior to use of the modified phage. In other embodiments, the phage may be produced with the biomolecular phage already bound or otherwise attached thereto, so it is ready to use immediately.

In particular embodiments, the invention includes a composition, e.g., a pharmaceutical composition, comprising a modified phage of the invention and one or more pharmaceutically acceptable carriers, excipients, or diluents. In certain embodiments, the composition comprises one or more buffer, cryoprotectant, stabilizer, lycoprotectant, or preservative.

In particular embodiments, a modified phage of the invention is dried, lyopholized, freeze-dried, and/or frozen for storage prior to use. In certain embodiments, the modified phage of the invention is stable for at least one month, at least two months, at least three months, at least four months, at least six months, at least one year, or at least two years when stored at either about 4° C. or about 20° to about 25° C. In certain embodiments, a modified phage is considered “stable” when at least 60%, at least 70%, at least 80%, at least 90%, at least 95%, at least 98% or at least 99% of the modified phage is capable of transducing a target cell after having been stored.

The invention further provides methods of producing methods of producing modified phage of the invention. In one embodiment, the invention includes a method of producing a genetically modified bacteriophage capable of delivering a therapeutic macromolecular polymer or an imaging agent to a target cell, comprising: (i) identifying a bacteriophage that expresses on its surface a targeting agent, e.g., a peptide, that binds to and/or mediates transduction of a target cell or microorganism, or genetically modifying a bacteriophage to express on its surface a targeting agent, e.g., a peptide, that binds to and/or mediates transduction of a target cell on the bacteriophage surface; and (ii) genetically modifying the bacteriophage to express one or more conjugation peptides on the phage surface. In particular embodiments, the one or more conjugation peptide is fused to one or more phage coat protein. In certain embodiments, the one or more phage coat protein is selected from the group consisting of: p3, p6, p7, p8, and p9. In one embodiment, the one or more phage coat protein comprises p8. In one embodiment, the one or more phage coat protein comprises p9. In certain embodiments, a first conjugation peptide is fused to p8 and a second conjugation peptide is fused to p9. In particular embodiments, at least one of said conjugation peptides is a sortase bridging domain or sortase recognition domain.

In various embodiments, step (i) comprises screening a plurality of bacteriophage to identify a bacteriophage capable of binding and/or transducing the target cell or microorganism. In particular embodiments, the plurality of bacteriophage is a library of genetically modified bacteriophage that express different exogenous peptides on the phage surface.

In related embodiments, the method further comprises coupling or binding the therapeutic macromolecular polymer, the imaging agent, or the peptide that binds an imaging agent to said genetically modified bacteriophage. In particular embodiments, the therapeutic macromolecular polymer, imaging agent, or peptide that binds an imaging agent comprises a coupling domain. In particular embodiments, the coupling domain is a sortase recognition domain. In particular embodiments, the sortase recognition domain is a sortase A or sortase B recognition domain.

The invention contemplates that therapeutic macromolecular polymers, imaging agents, and peptides that binds an imaging agent may be coupled to modified phage using protein coupling systems, such as the sortase coupling system. Certain coupling systems include two peptides or protein domains that bind to each other, wherein one may be fused to a portion of the phage and the other may be fused to the macromolecular polymer, imaging agent, or peptide that bind an imaging agent. Briefly, the sortase coupling system may be used to couple or fuse to polypeptides or peptides, wherein one of the two polypeptides or peptides to be coupled or fused, e.g., a phage coat protein, comprises a sortase bridging domain, and the other polypeptide or peptide to be coupled or fused, e.g., a therapeutic macromolecular biopolymer or imaging reagent, comprises a sortase recognition domain. In other embodiments, the phage coat protein comprises a sortase recognition domain, and the therapeutic macromolecular biopolymer or imaging reagent comprises a sortase bridging domain. In particular embodiments, the sortase coupling system comprises a sortase A recognition domain and a sortase A bridging domain, whereas in other embodiments, the sortase coupling system comprises a sortase B recognition domain and a sortase B coupling domain. In particular embodiments, the sortase A bridging domain comprises or consists of one or more glycine residues, e.g., (Gly)₂, (Gly)₃, (Gly)₄, or (Gly)₅, and a sortase A recognition domain comprises or consists of the amino acid sequence LPTGAA. In particular embodiments, the sortase B bridging domain comprises or consists of five alanine residues, and the sortase B recognition domain comprises or consists of the amino acid sequence LPTGGG. It is understood that either the sortase recognition domain or the sortase bridging domain may be fused to the phage protein, with the other domain fused to the macromolecular biopolymer or imaging agent.

In particular embodiments, the coupling step comprises contacting the genetically modified bacteriophage with the therapeutic macromolecular polymer, imaging agent, or peptide that binds an imaging agent, in the presence of a coupling agent. For example, in particular embodiments, where the one or more conjugation peptide comprises a sortase bridging domain, and the coupling domain comprises a sortase recognition domain, the coupling agent is a sortase, e.g., sortase A or sortase B.

Additional illustrative embodiments of genetically modified phage of the invention are described below in the general context of either therapeutic agents or imaging reagents. However, it is understood that any of the features of these illustrative phage may be incorporated into each other and remain within the scope of the invention.

Bacteriophage Genetically Modified for Delivery of Therapeutic Agents

Certain embodiments of the genetically modified bacteriophage of the invention are useful for delivering a therapeutic agent to a disease cell. In various embodiments, the therapeutic agent may be delivered in vitro, ex vivo, or in vivo. For example, in particular embodiments, a genetically modified bacteriophage comprising a therapeutic agent is delivered to a subject in need thereof, e.g., a subject comprising a disease cell, or a subject infected with a microorganism, such as a bacterium or a virus. In other embodiments, a genetically modified bacteriophage comprising a therapeutic agent is delivered to a disease cell that has been removed from subject in need thereof, and which may be returned to the subject.

Accordingly, in certain embodiments, the invention includes a genetically modified bacteriophage comprising: (1) a targeting agent, e.g., a peptide, that mediates transduction of a disease cell or binds to a microorganism, wherein said targeting agent is displayed on the phage surface; and (2) a therapeutic agent that is fused to or bound to a coat protein displayed on the phage surface. In particular embodiments, the genetically modified bacteriophage further comprises: (3) an endosomolytic peptide fused to a phage coat protein. In particular embodiments, the targeting peptide is fused to the p3 coat protein. In particular embodiments, the therapeutic agent is fused to or bound to the p8 coat protein, e.g., fused to p8 via a protease cleavable linker. In particular embodiments, the endosomolytic peptide is fused to the p9 coat protein.

In particular embodiments, the therapeutic agent is a macromolecular biopolymer, which includes but is not limited to: proteins, peptide, polynucleotides, and molecules comprising one or both of polynucleotides and proteins or peptides. Illustrative polypeptides include, but are not limited to, antibodies and fragments thereof, proteins, including both natural or synthetic proteins, therapeutic proteins, dominant negative proteins or peptides, aptamers, and nanobodies. Illustrative polynucleotides include, but are not limited to, single-stranded or double-stranded DNA or RNA or mixtures thereof, antisense RNA, short interfering RNA, microRNA, and polynucleotides encoding a therapeutic peptide or polypeptide, such as, e.g., a protein (natural or synthetic), antibody or fragment thereof, or nanobody.

In particular embodiments, the targeting peptide binds to a disease cell. Disease cells include but are not limited to tumor cells or cancer cells, inflammatory disease cells, neurological disease cells, cardiac disease cells, immune disorder cells, autoimmune disorder cells, infected cells, lysosomal storage disorder-affected cells, metabolic disease cells, or cells affected by any other disease or disorder. In certain embodiments, the infected cell is infected with a microorganism, such as a virus. In particular embodiments, the disease cell is a solid tumor cell or a liquid tumor cell. In particular embodiments, the tumor cell is malignant, while in other embodiments, it is benign. In certain embodiments, the tumor cell is a breast cancer cell, a prostate cancer cell, a lung cancer cell, a brain cancer cell, a kidney cancer cell, a liver cancer cell, a skin cancer cell, a leukemia cell, or a lymphoma cell. In certain embodiments, the prostate cancer cell is a human prostate specific membrane-bound antigen (PSMA)-negative cell with high metastatic potential.

In certain embodiments, the phage binds to a microorganism, such as, e.g., a bacterium or a virus.

In particular embodiments, the therapeutic agent is any known small molecule or macromolecular anti-tumor agent or a cytotoxic agent, or an agent used to treat any of a variety of different disease or disorders, including but not limited to tumors or cancers, inflammatory diseases, neurological diseases, cardiac diseases and disorders, immune disorders, autoimmune disorders, infections, lysosomal storage disorders, metabolic diseases or conditions, or any other disease or disorder. In certain embodiments, the therapeutic agent is an antimicrobial agent, such as an antiviral agent or an antibiotic. In particular embodiments, the therapeutic agent inhibits the growth or proliferation of the microorganism. In particular embodiments, it kills the microorganism.

In certain embodiments, the modified bacteriophage is used for replacement therapy, e.g., to deliver a macromolecular biopolymer that is missing in at least certain cells of a subject. For example, the subject may have a genetic disease wherein one or more normal proteins are not produced, are produced at a reduced level, or are produced in a mutant form. A modified bacteriophage may be used to deliver the normal protein, or a polynucleotide encoding the normal protein, to all or specific cells of the subject, e.g., cells that are adversely affected due to the lack of the normal protein. Thus, the modified bacteriphage may be used for gene therapies.

In particular embodiments, the disease or disorder is a solid tumor or a liquid tumor. In particular embodiments, the tumor is malignant, while in other embodiments, it is benign. In certain embodiments, the tumor is a breast cancer, a prostate cancer, a lung cancer, a brain cancer, a kidney cancer, a liver cancer, a skin cancer, a leukemia, or a lymphoma; however, all types of cancers can be targeted and treated according to the invention. In certain embodiments, the disease is an infection, such as a microbial infection, e.g., a viral infection or a bacterial infection.

In certain embodiments useful for therapeutic agent delivery applications, p3 is engineered to display a target cell or target microorganism specific binding agent, e.g., a peptide or PTD. As shown in the accompanying Examples, fusion of Ypep to p3 allowed phage to potently and selectively penetrate human prostate cancer cells in vitro. In related embodiments, a therapeutic agent (e.g., a peptide or protein) is displayed on p8, and engineered to only release therapeutic cargo from the host phage when that phage is in the cytosol of a target cell. Studies on the mechanism of phage uptake by human prostate cancer cells suggest an endocytotic process. Therefore, delivery of functional therapeutic cargo may require release from the endosome to the cytosol. In particular embodiments, disease cell-penetrating phage display an endosomolytic peptide on p9 (FIG. 1A).

In certain embodiments, the invention includes a genetically modified bacteriophage comprising: (1) a targeting agent comprising a PTD, wherein said PTD comprises Ypep or a Ypep mutant, and wherein said targeting agent is fused to or bound to a p3 coat protein displayed on the phage surface; and (2) an anti-tumor agent that is fused to or bound to a p8 coat protein displayed on the phage surface. In particular embodiments, the genetically modified bacteriophage further comprises: (3) an endosomolytic peptide fused to a p9 coat protein. In particular embodiments, the bacteriophage is used to treat a prostate cancer.

Bacteriophage Genetically Modified for Delivery of Imaging Reagents

In certain embodiments, the genetically modified bacteriophage of the invention are used to deliver or bind an imaging reagent to a disease cell, or to a microorganism. In various embodiments, the imaging reagent may be delivered in vitro, ex vivo, or in vivo. For example, in particular embodiments, a genetically modified bacteriophage comprising an imaging reagent is delivered to a subject in need thereof, e.g., a subject comprising a disease cell. Accordingly, in certain embodiments, the invention includes a genetically modified bacteriophage comprising: (1) a targeting agent (e.g., a peptide) that binds to and/or mediates transduction of a target cell (e.g., a disease cell) or microorganism (e.g., a virus), wherein said targeting agent is displayed on the phage surface; and (2) an imaging reagent, or a peptide that binds an imaging agent, that is fused to or bound to a coat protein displayed on the phage surface. In particular embodiments, the targeting peptide is fused to the p3 coat protein. In particular embodiments, the imaging reagent, or the peptide that binds the imaging agent, is fused to or bound to the p8 coat protein, e.g., fused to p8 via a protease cleavable linker.

A variety of imaging reagents are known and available in the art. Illustrative imaging reagents include, e.g., radioisotopes, fluorescent molecules, fluorescence and near infrared fluorescent proteins, e.g., and metals. In one embodiment, the imaging reagent is an iron-oxide nanoparticle. Peptides that bind imaging reagents are also known and available in the art. In one embodiment, the peptide that binds an imaging reagent is an iron-oxide nanoparticle-binding peptide. Examples include triglutamate, or an iron oxide nanoparticle binding peptide evolved from phage display (GSTTSLKY).

In particular embodiments, the targeting agent binds to a disease cell. Disease cells include but are not limited to tumor cells or cancer cells, inflammatory disease cells, neurological disease cells, cardiac disease cells, immune disorder cells, autoimmune disorder cells, infected cells, lysosomal storage disorder-affected cells, metabolic disease cells, or cells affected by any other disease or disorder. In particular embodiments, the disease cell is a solid tumor cell or a liquid tumor cell. In particular embodiments, the tumor cell is malignant, while in other embodiments, it is benign. In certain embodiments, the tumor cell is a breast cancer cell, a prostate cancer cell, a lung cancer cell, a brain cancer cell, a kidney cancer cell, a liver cancer cell, a skin cancer cell, a leukemia cell, or a lymphoma cell, although all cancer cells at various stages of growth can be targeted and treated according to the invention. In certain embodiments, the prostate cancer cell is a human prostate specific membrane-bound antigen (PSMA)-negative cell with high metastatic potential. In certain embodiments, the disease cell is a cell infected with a microorganism, such as, e.g., a virus. In certain embodiments, the targeting agent binds a microorganism, such as a bacterium or virus.

In one embodiment for targeted iron oxide nanoparticle MRI imaging reagent delivery, human prostate cancer cell-penetrating phage are genetically engineered to display an iron oxide nanoparticle-binding peptide on p8. Phage displaying these iron oxide nanoparticle-binding peptides can y be mixed with those nanoparticles to generate a phage-nanoparticle complex, which will carry those MRI imaging reagents to the targeted cell (FIG. 1B).

In certain embodiments, a genetically modified bacteriophage comprises (1) a targeting agent comprising a PTD, wherein said PTD comprises Ypep or a Ypep mutant, and wherein said targeting agent is fused to or bound to a p3 coat protein displayed on the phage surface; and (2) an iron oxide nanoparticle-binding peptide fused to a p8 coat protein displayed on the phage surface. In particular embodiments, the bacteriophage comprises an iron oxide nanoparticle MRI imaging agent bound to the iron oxide nanoparticle binding peptide. In particular embodiments, the bacteriophage is used to detect or locate prostate cancer cells in a subject, e.g., for MRI imaging or a prostate tumor or metastasis thereof.

### Methods of Using Engineered Bacteriophage

Modified phage of the invention may be used to deliver any macromolecular biopolymer to a cell or microorganism. Accordingly, in particular embodiments, modified phage of the invention are used to deliver a therapeutic agent to a cell or microorganism, e.g., to treat, inhibit, or prevent a disease, disorder or infection. In other embodiments, modified phage of the invention are used to deliver an imaging reagent to a cell or microorganism, e.g., for detecting the presence and location of diseased cells within a tissue, organ, or organism, e.g., a mammal.

In certain embodiments, a modified phage of the invention is used to deliver a macromolecular biopolymer to a cell. In various embodiments, the cell is isolated, or it is present in a tissue, an organ, or within a subject, e.g., a mammal. In related embodiments, the method comprises contacting a cell with a modified phage or composition of the invention, wherein said modified phage comprises a macromolecular biopolymer.

In certain embodiments, a modified phage of the invention is used to treat, prevent or inhibit a disease or disorder in a subject, e.g., a mammal, such as a human, in need thereof. In particular embodiments, a subject is in need of treatment when the subject has been diagnosed with, or is considered at risk of developing, the disease or disorder being treated, inhibited, or prevented. A subject may be treated in vivo or ex vivo. In related embodiments, the method comprises providing to a subject in need thereof an effective amount of a modified phage or composition of the invention, wherein said modified phage comprises a therapeutic agent useful for treatment of the disease or disorder. In certain embodiments, the disease or disorder is an infection, e.g., a bacterial infection or a viral infection.

In certain embodiments, a modified phage of the invention is used to deliver an imaging reagent to target cells, e.g., disease cells, or microorganisms, e.g., viruses, in a subject, e.g., a mammal, such as a human, in need thereof. In particular embodiments, a subject is in need of imaging when the subject has been diagnosed with, or is considered at risk of developing, the disease or disorder, e.g., an infection or tumor, being imaged. In related embodiments, the invention includes a method of determining whether a subject has a disease or disorder, or diagnosing a disease or disorder in a subject, comprising providing to the subject a modified phage of the invention that selectively or preferentially binds cells associated with the disease or disorder, or binds a microorganism associated with infection, and which comprises an imaging reagent, and then performing imaging to detect the presence or absence of the imaging reagent, thereby determining that the subject has or does not have the disease or disorder, wherein the detection of the imaging reagent indicates that the subject has the disease or disorder, and the absence of the imaging reagent indicates that the subject does not have the disease or disorder. In a related embodiment, the invention includes a method of determining the location of a disease or disorder, or cells associated with the disease or disorder in a subject, comprising providing to the subject a modified phage of the invention that selectively or preferentially binds cells associated with the disease or disorder, and which comprises an imaging reagent, and then performing imaging to detect the location of the imaging reagent within the subject, thereby determining the location of the disease or disorder, or cells associate with the disease or disorder within the subject. In particular embodiments, such methods may be used to determine if a cancer has metastasized to a location in the subject beyond the location of the primary tumor. In particular embodiments, such methods are used to determine the borders or margins of a tumor, in order to assist with surgical removal of the tumor.

In certain embodiments, the amount of or effective amount of a modified phage of the invention that is provided to a cell or a subject, is about, at least, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mg, mg/kg, or mg/m², or any range derivable therein. Alternatively, the amount may be expressed as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mM or M, or any range derivable therein.

In certain embodiments, the effective amount of a modified phage of the invention that is provided to a subject, comprises about, at least, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mg of therapeutic agent, mg therapeutic agent/kg subject, or mg therapeutic agent/m² subject, or any range derivable therein. Alternatively, the amount may be expressed as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mM or M, or any range derivable therein.

The amount of modified phage of the present invention to provide to a subject may be readily determined by a medical personnel based upon the use of the phage, the amount of therapeutic agent or imaging agent present in the modified phage, the route of delivery, and the nature of the subject and disease being treated or imaged.

According to various embodiments of the methods of the present invention, a subject, or tissue or organ thereof, is provided with a modified phage of the invention, e.g., intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, intraocularly, subcutaneously, subconjunctival, intravesicularly, mucosally, intrapericardially, intraumbilically, intraocularally, orally, topically, locally, by injection, by infusion, by continuous infusion, by absorption, by adsorption, by immersion, by localized perfusion, via a catheter, or via a lavage. In particular embodiments, it is provided parenterally, e.g., intravenously, or by inhalation.

The invention also provides a method of determining the presence of or an amount of a target cell within a plurality of cells, said method comprising contacting the plurality of cells with a modified phage that comprises a targeting agent, e.g., a peptide, that specifically or preferentially binds to the target cell and a coupled or bound imaging reagent, washing the phage from the plurality of cells, and then detecting the presence of or the amount of the imaging agent within the plurality of cells, thus determining the presence of or the amount of target cell within the plurality of cells. In a related embodiment, the invention provides a method of determining the presence of or an amount of a microorganism within a sample, e.g., a biological sample, such as a blood or urine sample, said method comprising contacting the biological sample with a modified phage that comprises a targeting agent, e.g., a peptide, that specifically or preferentially binds to the microorganism and a coupled or bound imaging reagent, washing the phage from the biological sample or microorganisms therein, and then detecting the presence of or the amount of the imaging agent associated with the biological sample or microorganism therein, thus determining the presence of or the amount of target microorganism within the biological sample.

### EXAMPLES

### Example 1

### In Vitro Evolution of a PC-3 Human Prostate Cancer Cell-Selective Penetrating Phage

Protein Transduction Domains (PTDs) are short peptides that promote the internalization of small and macroscale cargo. While relatively simple to prepare and use, unfortunately, common polycationic PTDs, such as the HIV-1 transactivator of transcription (Tat) peptide, the Drosophila Antennapedia-derived penetratin peptide, and polyarginine, do not selectively penetrate diseased cells. Moreover, their potency of cell-penetration can be quite low. This Example describes the evolution of a prostate cancer cell-selective penetrating phage and the identification of a novel prostate cancer cell-specific PTD.

### Evolution of Prostate CancerCell-Selective PTD

Following the scheme shown in FIG. 2, we performed rounds of positive selection on a phage library to evolve PTDs that penetrate PC-3 cells, a PSMA-negative human prostate cancer line with high metastatic potential. After three rounds, we enriched a linear twelve amino acid peptide, which we call “Ypep” (N-YTFGLKTSFNVQ-C). Ypep had tunable cell penetration potency and cell-selectivity profiles, which were tightly controlled by multivalency. The evolved M13 phage displayed five copies of Ypep on the N-terminus of p3. These (Ypep)₅-phage penetrated PC-3 cells with extreme potency (1.7 pM) and excellent cell selectivity.

To determine potency, PC-3 cells were grown to 80% confluency in a 6-well plate. Whole blood was diluted in half with F12K/10% FBS. To this solution, 1.3×10⁹ (Ypep)₅-phage (1.67 pM) were added. Cells were incubated in this solution for 3 hr at 37° C. under a 5% CO2 environment. Phage were titred, then diluted. Using avigadro's number, molarity was determined.

Ypep-dependent delivery was non-toxic under the conditions tested, and (Ypep)₅-phage penetrated PC-3 cells in the presence of untreated (active) human blood. Collectively, our findings revealed Ypep as a novel PTD with cell-selectivity and potency of cell uptake profiles that are controlled through multivalency effects.

Multivalency effects play crucial roles in various biological processes, and have been shown to control the mechanism of cellular uptake for a previously reported PTD. In order to measure the potency and cell-selectivity of Ypep-dependent cell penetration, as well as determine if any multivalency effects are associated with Ypep-dependent internalization, we treated cells with GFP containing a single N-terminal Ypep (Ypep-GFP), or N- and C-terminal Ypep (Ypep-GFP-Ypep). In many ways, GFP is an ideal protein to test the characteristics of a PTD, as well as interrogate multivalency effects. GFP is simple to express, stable, and is easy to image. While obviously not of human origin, the molecular weight (27 kDa) and net theoretical charge at physiological pH (−6) of GFP are within the average range among human proteins expressed in E. coli. Like most proteins, wild-type GFP does not penetrate human cells. GFP and its variants, or proteins with similar biophysical characteristics, have been used for various bioimaging applications. Finally, the N- and C-termini of GFP are in close proximity and have similar directionality, enabling multivalent interactions between a receptor and fusions to these termini.

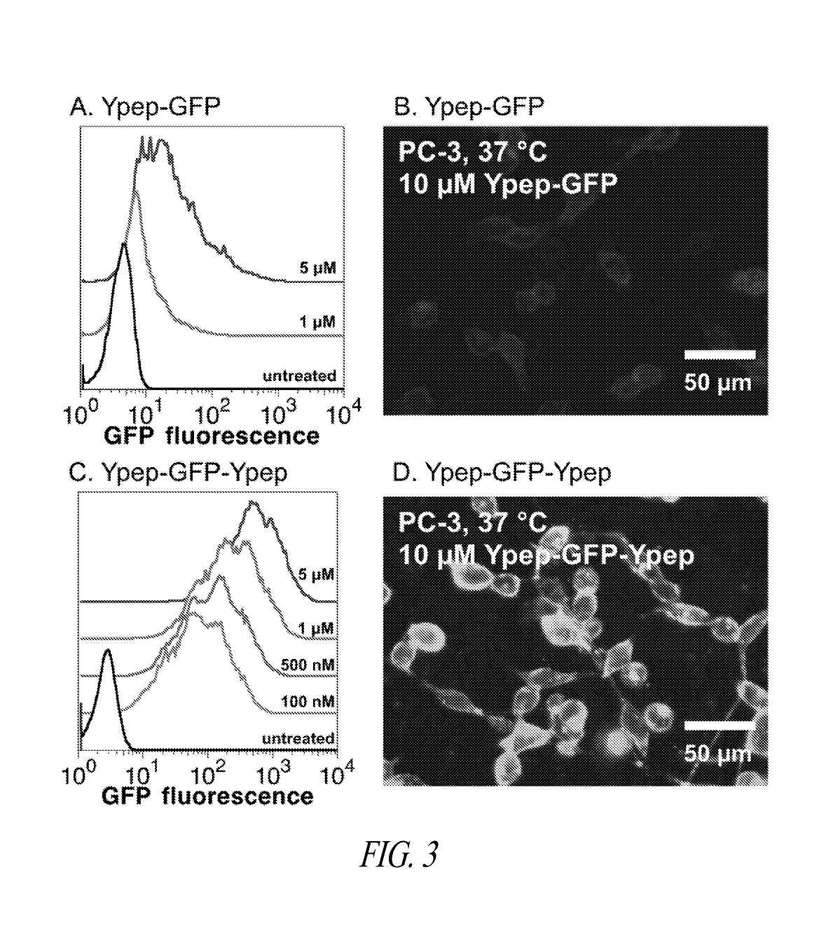

PC-3 cells were treated with Ypep-GFP or Ypep-GFP-Ypep solutions, then washed three times with a phosphate buffered saline (PBS) containing 20 U/mL heparin sulfate, which has been shown to remove surface-bound protein from mammalian cells. Internalized GFP levels were measured by flow cytometry. We observed a concentration-dependent increase in GFP levels for both fusion proteins; however, two copies of Ypep on GFP drastically increased the potency of GFP delivery (FIGS. 3A and 3C). A 24-fold increase in GFP fluorescence was observed in PC-3 cells treated with 0.5 μM Ypep-GFP-Ypep, compared to PC-3 cells treated with the same concentration of Ypep-GFP. PC-3 cells treated with 100 nM Ypep-GFP-Ypep had 3-fold higher GFP fluorescence compared to cells treated with 5 μM Ypep-GFP. (Ypep)₅-phage cell penetration is extremely potent. (Ypep)₅-phage penetrated PC-3 cells at concentrations as low as 1.7 pM. This value was obtained by taking the PFU (plaque forming units) and dividing by avigadro's number (6.02×1026) to calculate moles, then dividing that number by volume of dilutent in liters (L). Collectively, these data suggest an important role for multivalency in Ypep-dependent delivery. Flow cytometry data was supported by fluorescence microscopy, which showed much higher levels of internalized Ypep-GFP-Ypep in PC-3 cells following treatment with a 10 μM solutions of either Ypep-GFP or Ypep-GFP-Ypep (FIGS. 3B and 3D).

### Cell-Selectivity of Ypep-Dependent Delivery

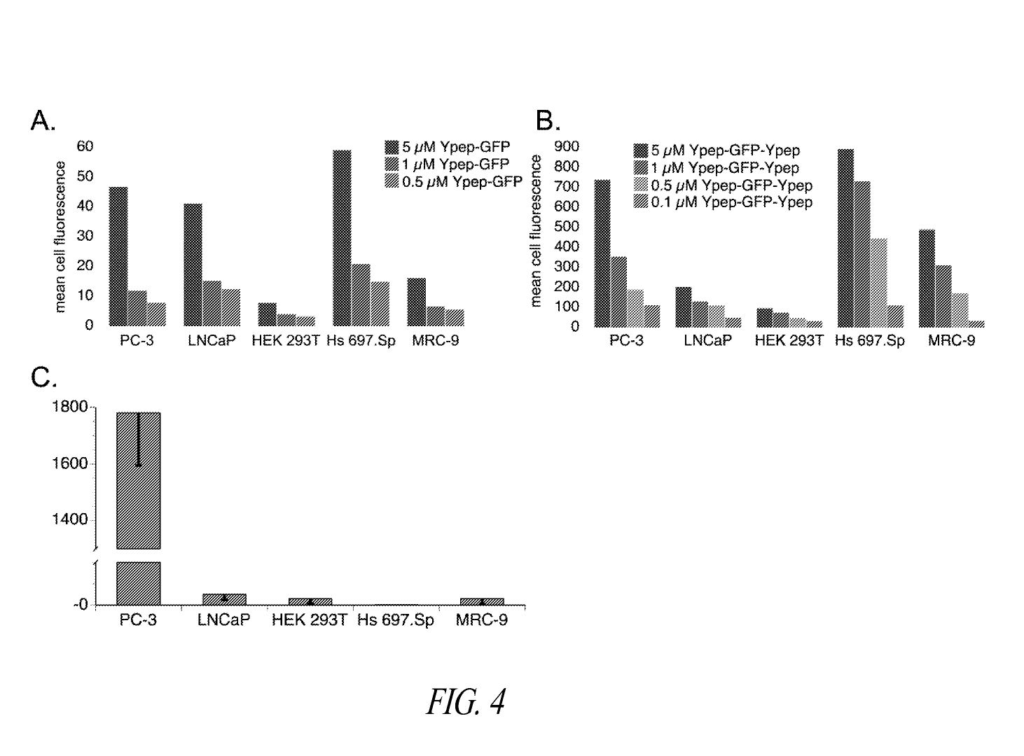

Given the role multivalency plays in the potency of Ypep-dependent delivery, we hypothesized that those same effects may contribute to the cell-selectivity of delivery. We compared the delivery of Ypep-GFP, Ypep-GFP-Ypep, and (Ypep)₅-phage in PC-3 human prostate cancer cells (PSMA-neg), LNCaP human prostate cancer cells (PSMA-pos), HEK-293T human embryonic kidney cells, MRC-9 human lung fibroblast cells, and Hs 697.Sp human spleen fibroblast cells. Cells were treated for 3 hours and washed to remove surface-bound phage. The potency and cell-selectivity of Ypep-GFP and Ypep-GFP-Ypep delivery was measured by flow cytometry. Phage titering from cell lysate was used to compare the amount of internalized phage in each cell line (FIG. 4).

For each treatment, the highest GFP levels were observed in PC-3 (PSMA-neg), LNCaP (PSMA-pos) prostate cancer cells, and Hs 697.Sp spleen cells (FIG. 4A), with the latter having the absolute highest GFP levels. Similar amounts of GFP was delivered to PC-3 and LNCaP cells, and Hs 697. Sp cells treated with 5, 1, or 0.5 μM Ypep-GFP had 1.3-, 1.7-, and 1.9-fold higher GFP fluorescence, respectively, compared to PC-3 cells. Ypep-GFP-Ypep has improved cell selectivity for penetration of PC-3 cells (FIG. 4B). For example, while Ypep-GFP indiscriminately penetrated PC-3 (PSMA-neg) and LNCaP (PSMA-pos) prostate cancer cells, PC-3 cells treated with either 0.5, 1, or 5 μM Ypep-GFP-Ypep were 3.2-, 3.5-, and 2.7-fold more fluorescent than LNCaP (PSMA-pos) cells treated under the same conditions. However, similar to the results for Ypep-GFP, Hs697.Sp cells received the highest absolute levels of Ypep-GFP-Ypep (FIG. 4B). Nonetheless, the three-fold increase in selectivity for PC-3 cells over LNCaP cells suggests an important role for multivalency in the cell-selectivity of delivery.

We next tested the selectivity of (Ypep)₅-phage delivery in PC-3, LNCaP, HEK-293, MRC-9, and Hs 697.Sp cells by comparing the titer of phage from lysate of each cell after incubation and washing steps. Cells were treated with a F12K/10% FBS solution containing 1.7 pM (Ypep)₅-phage. After incubating the phage with cells, cell surface-bound phage was removed with an extensive washing protocol. No phage was found in any of the final washing solution, suggesting that all cell surface-bound phage were removed. (Ypep)₅-phage potently penetrated PC-3 prostate cancer cells (PSMA-neg) (FIG. 4C). In three separate experiments, an average of 1780 (±243) (Ypep)₅-phage plaque-forming units per milliliter (pfu/mL) were generated from PC-3 cell lysate. In contrast, appreciable levels (Ypep)₅-phage were not observed in the lysate of LNCaP (PSMA-pos), HEK-293T (kidney), Hs 697.Sp (spleen), and MRC-9 (lung) cells (FIG. 4C). PC-3 cells treated with phage that did not contain Ypep did not have phage in cell lysate. Pfu/mL is a functional measurement rather than a measurement of the absolute quantity of internalized phage; viral particles that are defective and cannot infect E. coli, or which fail to infect E. coli did not produce a plaque and thus were not counted. Therefore, these numbers represent a lower limit for cell penetration by (Ypep)₅-phage. Collectively, these data represent a dramatic change in the potency and specificity of Ypep-dependent delivery, clearly demonstrate the critical role multivalency effects play in Ypep-dependent cell-selective penetration, and establish (Ypep)₅-phage as an extremely potent and PC-3 cell-selective cell-penetrating vehicle.

### Mechanistic Analysis of Ypep-Dependent Cell Penetration of PC-3 Cells

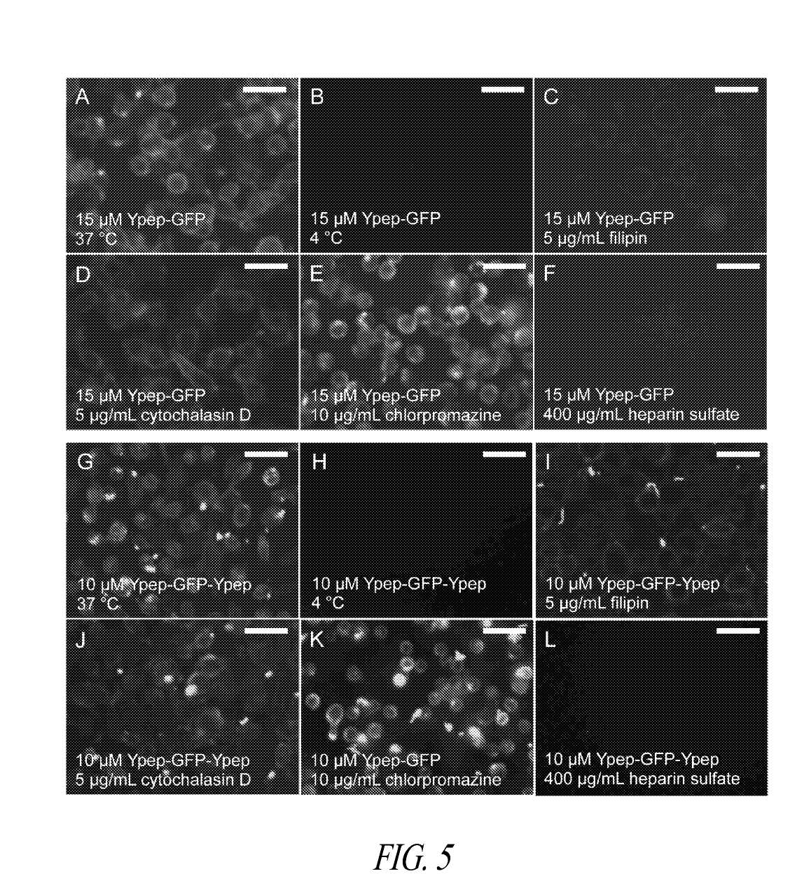

Unlike experiments conducted at 37° C., cell penetration was not observed when PC-3 cells were cooled to 4° C. before and during treatment with either Ypep-GFP or Ypep-GFP-Ypep (FIGS. 5B and 5H, respectively). These results suggest that cell penetration by these fusions requires an energy-dependent process, consistent with endocytosis. We next evaluated the cell penetration of each Ypep variant under conditions that block a particular component of an endocytotic pathway. PC-3 cells were incubated with relatively high concentrations of Ypep-GFP or Ypep-GFP-Ypep, and images for short periods of time, to assure that changes in cell fluorescence were due to inhibition of cell penetration as opposed to relatively small changes in uptake of low concentrations of GFP fusions that may or may not be associated with inhibition of endocytosis. When PC-3 cells were pre-treated with 5 μg/mL filipin, a small molecule known to inhibit lipid-raft/caveolae-dependent endocytosis, much lower cell fluorescence was observed compared to cells that were not treated with the inhibitor molecule (FIGS. 5C and 5I, respectively). In addition, pre-treating cells with 5 μg/mL cytochalasin D, an actin polymerization inhibitor, significantly decreased cell penetration of both GFP fusions (FIGS. 5D and 5J, respectively). In contrast, similar treatment with 10 μg/mL chlorpromazine, an inhibitor of clathrin-mediated endocytosis, had little effect on the efficiency of cell penetration for both Ypep-GFP and Ypep-GFP-Ypep (FIGS. 5E and 5K, respectively). Internalization of caveolae requires disruption of the local actin cytoskeleton. Therefore, inhibition of cell penetration as a result of lipid-raft/caveolae-dependent endocytosis and actin polymerization both support the conclusion that Ypep-GFP and Ypep-GFP-Ypep penetration of PC-3 cells was mediated by lipid-raft/caveolae-dependent endocytosis. Internalization of both Ypep-GFP and Ypep-GFP-Ypep was inhibited by 400 μg/mL heparin sulfate, (FIGS. 5F and 5L, respectively). Based on previous studies, this suggests that internalization required interaction(s) with one or more PC-3 cell surface glycosaminoglycans.

Unlike Ypep-GFP and Ypep-GFP-Ypep fusions, which can be removed from the cell surface using a relatively simple washing procedure, and for which internalized protein can be immediately imaged, experiments with phage require a relatively substantial and lengthy washing procedure in order to assure that all phage are removed from the cell surface and only internalized phage are isolated. Following treatment with filipin, cytochalasin D, and chloropromazine, PC-3 cells did not withstand the washing protocol. We observed significant loss of cells throughout the experiment due to cytotoxicity. Therefore, we were unable to obtain meaningful data from those experiments. Studies on the mechanism of (Ypep)₅-phage penetration in PC-3 cells were limited to conditions not cytotoxic to the cells during the course of the experiment. Unlike experiments conducted at 37° C., which yielded high levels of phage from cell lysate, no phage were isolated when PC-3 cells were cooled to 4° C. before and during treatment with (Ypep)₅-phage (data not shown). This result suggests that cell penetration of (Ypep)₅-phage requires an energy-dependent process, consistent with endocytosis. In addition, internalization of (Ypep)₅-phage was inhibited by heparin sulfate (data not shown), suggesting that internalization requires interaction(s) with one or more PC-3 cell surface glycosoaminoglycans. Collectively, and important to aspects of this proposal, all data indicated that Ypep-dependent delivery proceeds through endocytosis.

### Cytotoxicity and Robustness of Ypep-Dependent Delivery

To assess the cytotoxicity of all Ypep variants, we performed MTT assays on PC-3 cells after treatment with 0.5, 1, or 5 μM Ypep-GFP or Ypep-GFP-Ypep, or 1 nM (Ypep)₅-phage, which is 1,000-fold more concentrated than treatment solutions. These assays revealed no apparent cytotoxicity to PC-3 cells for any of the Ypep variants (data not shown). In order for a PTD to be used in vivo, it must penetrate the target cell in the presence of blood. We treated PC-3 cells with either 10 μM Ypep-GFP or Ypep-GFP-Ypep for three hours in F12K/10% FBS solution containing 50% human untreated (active) blood. Cells were then washed, and red blood cells were removed using standard methods. Cell fluorescence was measured by flow cytometry. Ypep-GFP-Ypep, but not Ypep-GFP, penetrated PC-3 cells in a solution containing human blood (data not shown). In addition, when PC-3 cells were treated with 1.7 pM (Ypep)₅-phage in a F12K/10% FBS solution containing 50% human blood, >500 pfu/mL were found in PC-3 cell lysate. Collectively, these data show that multivalent Ypep cell penetration proceeded in solutions containing human blood.

### Example 2

### Acute Toxicity and Biodistribution of (Ypep)₅-Phage In Vivo

To further assess the usefulness of (Ypep)₅-phage in for delivering biopolymers to cells in vivo, the biodistribution and acute toxicity of (Ypep)₅-phage was examined in mammals.

### Acute Toxicity of (Ypep)₅-Phage in Mice

Three sets of three ICR mice were obtained from National Cancer Institute. The mice were intravenously injected with 5 nM (Ypep)₅-phage (100 μL per 25 g mouse) diluted in Tris-buffered saline. The mice were then weighed and observed daily. No abnormalities were observed in the health of the mice. After 9 days, a challenge injection was administered with 5 nM Ypep-phage (100 μL per 25 g mouse). The mice were weighed and monitored for an additional 3 days. No signs of toxicity were observed during that time. Weight loss or gain was within ±3.4% before treatment.

The mice were then euthanized, and the heart, lungs, thymus, trachea, esophagus, peribronchial and peripancreatic lymph nodes, kidney, spleen, liver, gall bladder, small intestine, large intestine, and adrenal gland were removed. The tissues were preserved in formalin for histopathological analysis. All tissues were judged as normal or having incidental findings that are common in mice. There was no evidence of lesions induced by treatment of these mice. Taken together, these data show that (Ypep)₅-phage is not appreciably toxic to mice and does not elicit an appreciable immune response.

### Biodistribution of (Ypep) 5-Phage in Mice

Three nude mice were obtained from the National Cancer Institute. Mice were treated with 100 nM (Ypep)₅-phage diluted in Tris-buffered saline. The mice were intravenously injected with 100 μL of sample per 25 g mouse and sacrificed 4 hours post-injection. Whole blood was collected with cardiac puncture and liver, spleen, kidney, heart, lungs, brain, and small intestines were collected and flash frozen in liquid nitrogen. The tissue was stored at −80° C. until analysis.

To prepare tissue homogenates, the samples were thawed on ice and the half the tissue added to a 10 mL tissue grinder. A solution of 200 μL Tris-buffered saline containing complete ULTRA protease inhibitor cocktail was added to the tissue and the tissue was ground until a homogenous mixture was obtained. The cells were then lysed by adding 20 μL of mammalian cell lysis buffer (2% sodium deoxycholate, 10 mM Tris-HCl, 2 mM EDTA) to 20 μL of tissue homogenate. The solution was mixed vigorously and incubated at room temperature for 1 hour. The lysate solution was then titered with ER2738 E. coli and plaques were counted.

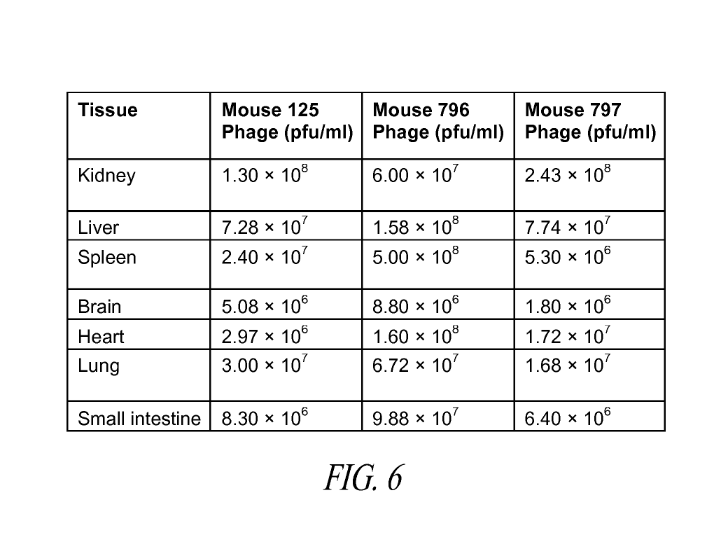

As shown in FIG. 6, (Ypep)₅-phage had good biodistribution, there were similar levels of phage in all tissue, and was not preferentially sequestered by the liver or kidney, not was it retained within the vasculature or excreted quickly. Taken together, these data show that (Ypep)₅-phage will have the opportunity to seek out and penetrate a PC-3 tumor when injected into mice. It is worth noting that the mice were treated with concentrations of phage that are much higher than needed for potent penetration in vitro. In addition, tissue was't washed to remove any unbound phage, so it is unclear if phage penetrated these tissues or simply bound to the exterior of those cells. These data only show that broad biodistribution is obtained in wild-type mice, which is desirable.

### Example 3

### (Ypep)₅-Phage Directed Delivery of a Nanoparticle Conjugate In Vitro

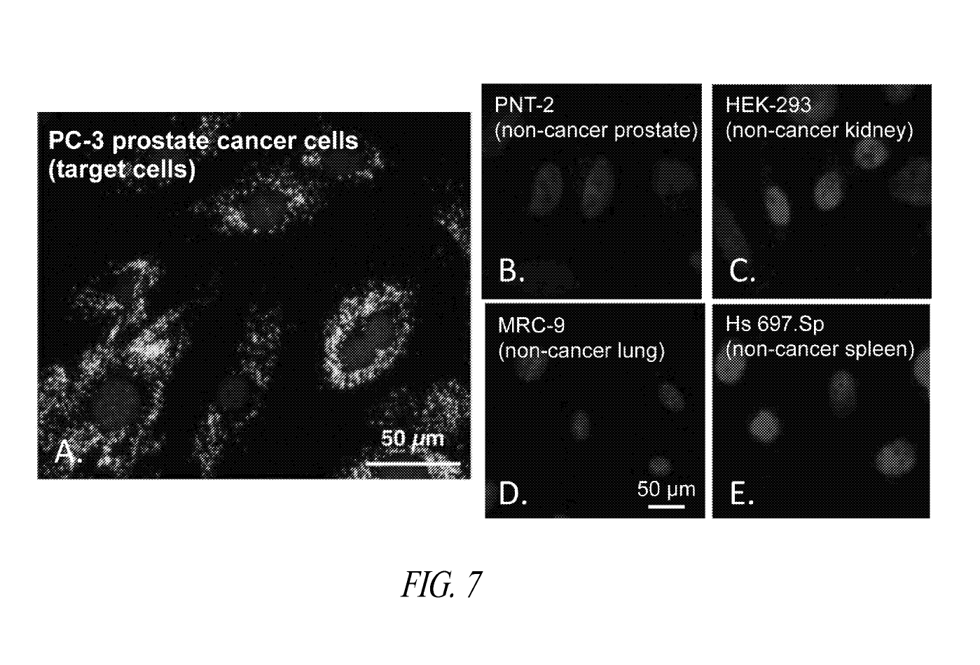

The ability of (Ypep)₅-phage to deliver a conjugated nanoparticle was demonstrated in PC-3 Cells. In order to see if a relatively large cargo conjugated to (Ypep)₅-phage altered the potency or selectivity of cell penetration and delivery, we engineered (Ypep)₅-phage to display a (GGS)₂ linker with a N-terminal lysine, and conjugated this phage to poly-N-acryloxylsuccinimide nanoparticles. These particles were concomitantly conjugated with GFP, which enabled their visualization. As seen in FIG. 7, particles conjugated to (Ypep)₅-phage penetrated PC-3 (target) cells, but did not penetrate a host of off-target cells. Collectively, these data show: (1) (Ypep)₅-phage delivers nanoscale objects selectively to PC-3 cells; and (2) conjugation between a p8 peptide on phage and a nanoscale object does not alter the cell-selectivity of (Ypep)₅-phage-dependent cell penetration.

### Example 4

### Identification of Ypep Mutants with Enhanced Uptake Efficiency

Ypep mutants with increased uptake efficiency and/or increased prostate cell selectivity were developed by mutating the original Ypep peptide. These mutants deliver higher levels of functional enzyme to human prostate cells.

To assess the specific contribution each Ypep residue plays in cell uptake efficiency, we made a library of Ypep alanine mutants and expressed these peptides as N-terminal fusions to GFP. PC-3 cells were treated with 5 μM of each Ypep-GFP mutant, a concentration previously shown to be sufficient for appreciable Ypep-GFP uptake. Cells were then exhaustively washed using conditions previously shown to remove cell surface-bound protein. The amount of internalized GFP was measured by flow cytometry. As seen in FIG. 8B, most mutationsresulted in significantly lower GFP delivery. However, Ypep-Gly4Ala and Ypep-Thr7Ala delivered ˜3.8- and ˜6.8-fold more GFP to PC-3 cells compared to native Ypep, respectively (FIG. 8B).

While most commonly used PTDs such as Tat, polyarginine and penetratin, which do not exhibit cell-selectivity, are polycationic, Ypep has a theoretical net change of +1. Interestingly, mutating the single positively charged residue (Lys6) to alanine decreased GFP uptake ˜4-fold (FIG. 8B). Based on these findings, we prepared a focused library of mutants with molecularly diverse residues at position 4 or 7. Ypep mutants containing either negatively charged (aspartic acid), positively charged (lysine), aromatic (phenylalanine), hydrogen bond donating (serine), or amide (asparagine) functional groups at positions 4 or 7 were expressed as N-terminal fusions to GFP. As seen in FIG. 8C, the Gly4Asp mutant exhibited significantly lower uptake, and Gly4Phe and Gly4Ser mutants achieved only slightly higher uptake than Ypep-GFP. However, Gly4Lys and Gly4Asn mutants were significantly improved. Ypep-GFP fusions containing Gly4Lys or Gly4Asn mutations delivered ˜3.2- and ˜19.2-fold more GFP to PC-3 cells, compared to native Ypep-GFP. Interestingly, small changes to asparagine 4 significantly lowered uptake. While the Gly4Gln mutant was ˜6.6-fold improved over Ypep, it was ˜2.8-fold less efficient than the Gly4Asn mutant. The fact that small structural changes at this position significantly lower uptake suggests that selective peptide-cell surface receptor interaction analogous to a selective protein—small molecule drug interaction facilitates uptake.

We next performed identical experiments to optimize residue 7. The Thr7Asp mutant exhibited essentially identical uptake efficiency as native Ypep. However, Thr7Lys, Thr7Ser, and Thr7Asn mutants all showed significantly lower transduction efficiencies. In contrast, the Thr7Phe mutant was significantly improved, and was able to deliver ˜7.6-fold more GFP to PC-3 cells, compared to native Ypep (FIG. 8D). Based on this finding, we measured uptake efficiencies for Ypep variants containing all possible proteinogenic aromatic residues at position 7 (FIG. 8D, blue bars). While both Thr7Tyr and Thr7Trp mutants significantly outperformed native Ypep, delivering ˜6.8- and ˜7.1-fold more GFP, respectively, neither outperformed the Thr7Phe mutant. In contrast, the Thr7His mutant showed significantly lower cell uptake compared to Thr7Tyr and Thr7Trp mutants, as well as native Ypep. Taken together, the reduced transduction we observed for the Thr7His and Thr7Lys mutants suggest that residues with positive charge, or partial positive charge, may not be tolerated at this position.

Additive effects play prevalent roles in many biological processes. We questioned whether combining beneficial single mutations might result in increased uptake efficiency. In order to characterize possible additive mutational effects, we prepared four Ypep double mutants that contain combinations of the most active single mutations at residues 4 and 7. Ypep double mutants that contain alanine, lysine, or asparagine at positive 4, and phenylalanine at position 7 were expressed as N-terminal fusions to GFP and added to PC-3 cells as previously described. The Gly4Ala:Thr7Phe, Gly4Lys:Thr7Phe, and Gly4Asp:Thr7Phe double mutants were found to be ˜3.5-, ˜5.6-, and ˜6.5-fold more efficient at GFP transduction than Ypep-GFP, respectively FIG. 8E). However, none of the double mutants exhibited higher uptake compared to the single mutant Ypep variants from which they were derived.

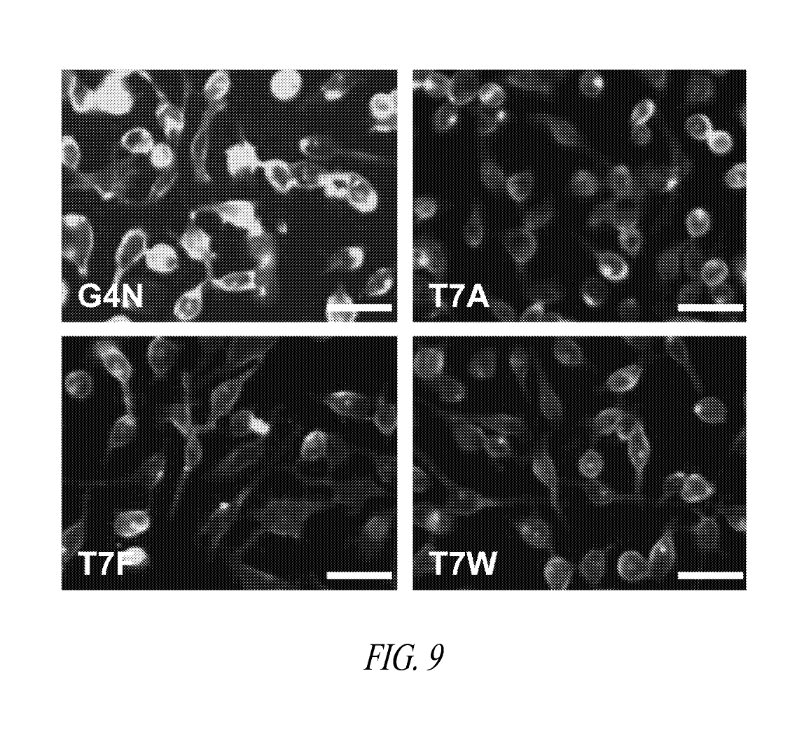

Based on these data, the Gly4Asn, Thr7Phe, Thr7Trp, and Thr7Ala mutants (colored blue (dark) in FIGS. 1C and 1D) were most improved over Ypep, with increased transduction efficiencies of 19.2-, 7.6-, 7.1-, and 6.8-fold, respectively. GFP uptake was confirmed for these four mutants by live-cell fluorescence microscopy. While only a very small amount of internalized GFP was observed in PC-3 cells following treatment with 5 μM Ypep-GFP (data not shown), large amounts of internalized GFP was observed in cells following treatment with the same concentration of the four most active Ypep(mutant)-GFP fusions (FIG. 9). Consistent with our flow cytometry data, the Gly4Asn mutant delivered the highest amount of GFP to the cell interior.

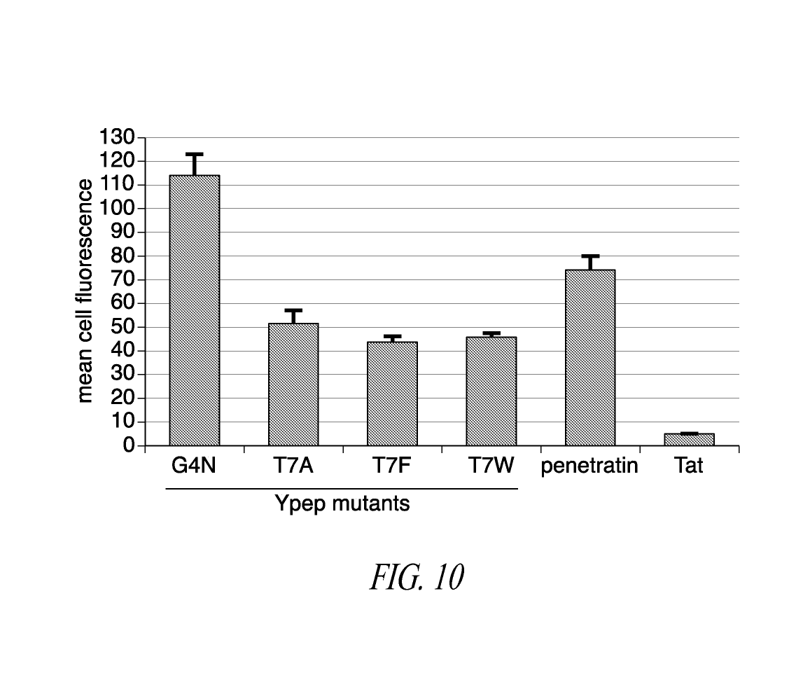

Tat and penetratin are both polycationic commercially available PTDs, which exhibit no appreciable cell-selectivity. In order to characterize the potential commercial utility of Ypep mutants for PC-3 cell-selective transduction, we compared uptake efficiencies for Tat-GFP, penetratin-GFP, and the four best Ypep variants identified as a result of mutagenesis studies. As shown in FIG. 10, following treatment with 1 μM PTD-GFP fusion, and washing, all Ypep mutants delivered significantly more GFP to the interior of PC-3 cells, compared to penetratin-GFP and Tat-GFP fusions. Most notably, uptake efficiency in PC-3 cells treated with 1 μM Ypep(Gly4Asn)-GFP was ˜1.5-fold and 23-fold higher than cells treated with either penetratin-GFP or Tat-GFP fusions, respectively. Interestingly, we were unable to express appreciable amounts of soluble (Arg)₉-GFP, suggesting limitations to polyarginine-based approaches to protein transduction.

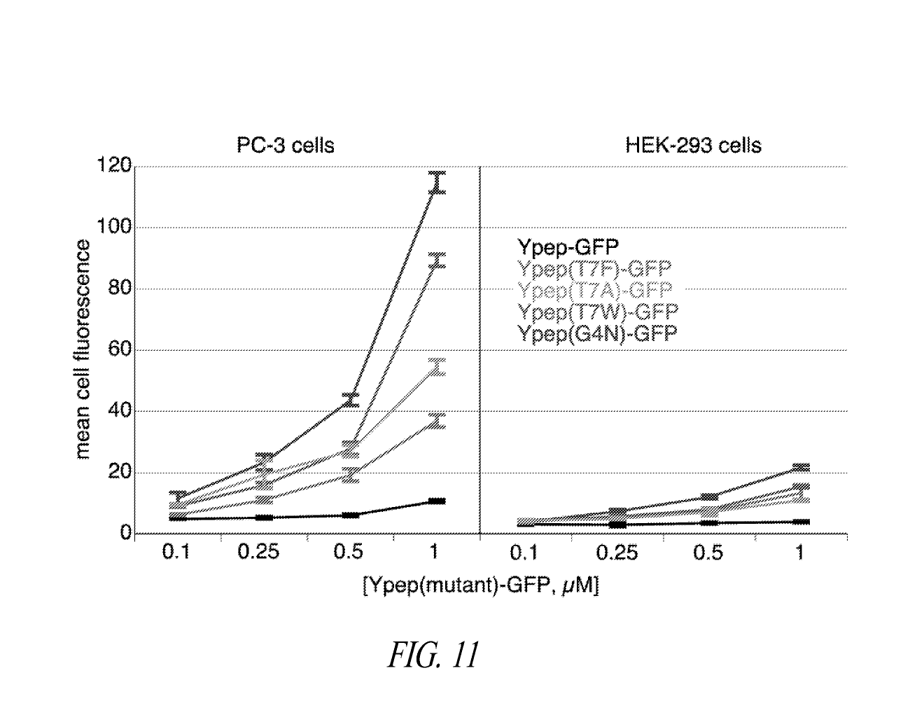

While mutational studies on Ypep resulted in the identification of variants with improved transduction efficiency, the effect of these mutations on cell-selectivity was unclear. In order to characterize the cell-selectivity of our most efficient Ypep mutants we compared GFP uptake in PC-3 (target) and off-target non-cancer human embryonic kidney cells (HEK-293). Cells were treated with 0.1-1 μM PTD-GFP fusion, washed as previously described to remove cell surface-bound material, and levels of internalized GFP was measured by flow cytometry. As shown in FIG. 11, many of the most efficient Ypep mutants also exhibited increased selectivity for PC-3 human prostate cancer cells. Consistent with our previous findings, Ypep delivered ˜1.6-, ˜1.8-, ˜1.7-, or 2.8-fold more GFP to PC-3 cells compared to HEK-293 cells, following treatment with 0.1, 0.25, 0.5, or 1 μM solutions, respective. While the Thr7Phe mutant exhibited similar selectivity for PC-3 cells (˜1.6, ˜2.0, ˜2.6, and ˜1.7-fold following 0.1-1 μM treatment), the Gly4Asn, Thr7Trp, and Thr7Ala mutants were significantly more selective for PC-3 cells. For example, Gly4Asn, Thr7Trp, and Thr7Ala Ypep mutants were ˜5.3, ˜5.8, and ˜5.0-fold more selective for PC-3 prostate cancer cells compared to HEK-293 cells. Taken together, these studies demonstrate a significant improvement in both the transduction efficiency and PC-3 cell-selectivity of multiple Ypep mutants found as a result of these studies.

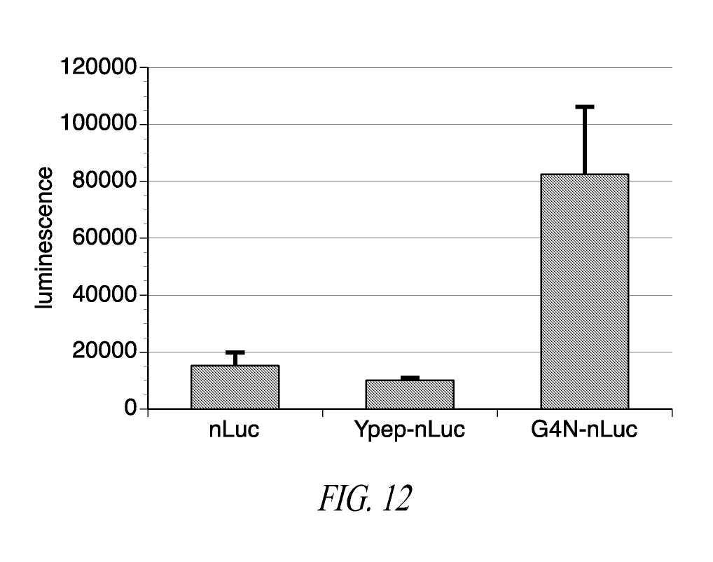

A further test of a PTD is intracellular delivery of a functional enzyme. Luciferase is a class of enzymes that oxidatize a photon-emitting substrate, resulting in bioluminescence. These enzymes enjoy extensive use as reporters and bioimaging reagents because of their ability to provide highly sensitive quantitation with broad linearity. Nanoluciferase (nLuc) is a recently reported ˜19 kDa variant of the small luciferase subunit from deep sea shrimp Oplophorus gracilirostris. As a simple test for functional intracellular enzyme delivery, we measured luciferase activity in PC-3 cells following treatment with nLuc or a Ypep(G4N)-nLuc fusion. Similar to the overwhelming majority of proteins, nLuc does not efficiently penetrate PC-3 cells. Cells treated with nLuc, washed to remove surface-bound protein, and treated with furimazine, a nLuc substrate, did not exhibit appreciably higher luminescence than untreated cells (FIG. 12). In addition, similar to our previous findings, relatively modest luminescence was observed in cells following treatment with Ypep-nLuc and furimazine (FIG. 12). In contrast, however, cells treated with Ypep(Gly4Asn)-nLuc exhibited substantially higher luminescence compared to cells treated with either nLuc or Ypep-nLuc. These findings suggest that appreciable levels of enzymatically active Ypep(Gly4Asn)-nLuc was delivered to the interior of PC-3 cells (FIG. 12).

Ypep, a prostate cancer cell-selective PTD, demonstrated uptake efficiency and cell-selectivity profiles that are dependent upon multivalency effects. When a single copy of Ypep was fused to GFP, modest uptake efficiency and cell selectivity was observed. Fusion of a single Ypep to the N-terminus of nLuc did not deliver appreciable functional protein to PC-3 cells. Mutational studies have identified a number of Ypep variants with significantly improved protein transduction. For example, a single mutation to Ypep (Gly4Asn) resulted in a variant with ˜19.2-fold higher transduction efficiency and ˜2-fold better selectivity for PC-3 cells over off-target HEK-293 cells. In contrast to Ypep, Ypep(Gly4Asn) delivered appreciable levels of nanoluciferase (nLuc) to the interior of PC-3 cells. Taken together, the findings demonstrate the functional utility of Ypep-dependent delivery of exogenous proteins to the interior of PC-3 prostate cancer cells. Our data suggest that the Ypep mutants described herein are well suited to serve as reagents for cell-selective (e.g., PC-3 cells) delivery of imaging and enzymatic proteins for basic research and biomedical applications.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention.

1. A genetically modified bacteriophage, said bacteriophage comprising:
(i) a targeting agent that binds to and/or mediates transduction of a target cell, wherein the targeting agent is displayed on the phage surface; and
(ii) one or more conjugation peptide displayed on the phage surface.

2. The genetically modified bacteriophage of claim 1, wherein said targeting agent is fused to a bacteriophage coat protein, or bound to a bacteriophage coat protein.

3. The genetically modified bacteriophage of claim 2, wherein said bacteriophage coat protein is selected from the group consisting of; p3 p6, p7, p8 and p9.

4-7. (canceled)

8. The genetically modified bacteriophage of claim 1, wherein said targeting agent is a biopolymer, optionally a peptide, protein, antibody or fragment thereof, nanobody or nucleic acid.

9. The genetically modified bacteriophage of claim 8, wherein said targeting agent is a peptide that comprises or consists of an amino acid sequence having a least 90%, at least 95%, or at least 99% identity to the amino acid sequence set forth in SEQ ID NO:1 or a peptide that comprises or consists of the amino acid sequence set forth in any one of SEQ ID NOs:1-32.

10-12. (canceled)

13. The genetically modified bacteriophage of claim 1, wherein said one or more conjugation peptide comprises one or more sortase bridging domain.

14. (canceled)

15. The genetically modified, bacteriophage of claim 1, wherein said one or more conjugation, peptide is fused to one or more phage coat protein.

16. The genetically modified bacteriophage of claim 15, wherein said one or more phage coat protein is selected from the group coexisting of: p3, p6, p7, p8 and p9.

17-20. (canceled)

21. The genetically modified bacteriophage of claim 1, wherein a therapeutic macromolecular biopolymer, an imaging reagent, or a peptide that binds an imaging reagent is conjugated to one of said one or more conjugation peptide.

22-51. (canceled)

52. A method of delivering a therapeutic macromolecular biopolymer or an imaging reagent to a cell or to a subject in need thereof, said method comprising contacting said cell with the bacteriophage of claim 21 or providing to said subject the bacteriophage of claim 21.

53. The method, of claim 52, wherein said method is performed in vitro, in vivo or ex vivo.

54-55. (canceled)

56. The method of claim 52, wherein said subject has been diagnosed with or is considered at risk of having a disease.

57. The method of claim 56, wherein said disease is a tumor.

58. The method of claim 57, wherein said turner is a prostate tumor.

59-61. (canceled)

62. A method of treating or preventing a disease in a subject in need thereof, comprising providing to the subject an effective amount of the bacteriophage of claim 21, wherein said bacteriophage comprises a coupled therapeutic macromolecular polymer.

63. The method of claim 62, wherein the disease is a tumor.

64. The method of claim 63, wherein the tumor is a prostate cancer.

65. (canceled)

66. A method of producing a genetically modified bacteriophage capable of delivering a therapeutic macromolecular polymer or an imaging agent to a target cell, said method comprising;
(i) identifying a bacteriophage that express on its surface a targeting agent, optionally a peptide, that mediates transduction of a target cell on the bacteriophage surface, or genetically modifying a bacteriophage to express on its surface a targeting agent, optionally a peptide, that mediates transduction of a target cell on the bacteriophage surface; and
(ii) genetically modifying the bacteriophage to express one or more conjugation peptides on the phage surface.

67-79. (canceled)

80. An isolated peptide comprising or consisting of an amino acid sequence having at least 90%, at least 95%, at least 98%, or at least 99% identity to the amino acid sequence set forth in SEQ ID NO:1.

81. The isolated peptide of claim 80, wherein said peptide comprises or consists of the amino acid sequence set forth in any one of SEQ ID NOs:1-32.

82-90. (canceled)


SEQUENCE LISTING (32 sequences)

SEQ ID NO: 1 - protein, 12 aa, Unknown
  Peptide identified by panning multiple bacteriophage
YTFGLKTSFNVQ

SEQ ID NO: 2 - protein, 12 aa, Artificial Sequence
  Mutated Ypep sequence
ATFGLKTSFNVQ

SEQ ID NO: 3 - protein, 12 aa, Artificial Sequence
  Mutated Ypep sequence
YAFGLKTSFNVQ

SEQ ID NO: 4 - protein, 12 aa, Artificial Sequence
  Mutate Ypep sequence
YTAGLKTSFNVQ

SEQ ID NO: 5 - protein, 12 aa, Artificial Sequence
  Mutated Ypep sequence
YTFALKTSFNVQ

SEQ ID NO: 6 - protein, 12 aa, Artificial Sequence
  Mutated Ypep sequence
YTFGAKTSFNVQ

SEQ ID NO: 7 - protein, 12 aa, Artificial Sequence
  Mutated Ypep sequence
YTFGLATSFNVQ

SEQ ID NO: 8 - protein, 12 aa, Artificial Sequence
  Mutated Ypep sequence
YTFGLKASFNVQ

SEQ ID NO: 9 - protein, 12 aa, Artificial Sequence
  Mutated Ypep sequence
YTFGLKTAFNVQ

SEQ ID NO: 10 - protein, 12 aa, Artificial Sequence
  Mutated Ypep sequence
YTFGLKTSANVQ

SEQ ID NO: 11 - protein, 12 aa, Artificial Sequence
  Mutated Ypep sequence
YTFGLKTSFAVQ

SEQ ID NO: 12 - protein, 12 aa, Artificial Sequence
  Mutated Ypep sequence
YTFGLKTSFNAQ

SEQ ID NO: 13 - protein, 12 aa, Artificial Sequence
  Mutated Ypep sequence
YTFGLKTSFNVA

SEQ ID NO: 14 - protein, 12 aa, Artificial Sequence
  Mutate Ypep sequence
YTFALKTSFNVQ

SEQ ID NO: 15 - protein, 12 aa, Artificial Sequence
  Mutated Ypep sequence
YTFDLKTSFNVQ

SEQ ID NO: 16 - protein, 12 aa, Artificial Sequence
  Mutated Ypep sequence
YTFFLKTSFNVQ

SEQ ID NO: 17 - protein, 12 aa, Artificial Sequence
  Mutated Ypep sequence
YTFKLKTSFNVQ

SEQ ID NO: 18 - protein, 12 aa, Artificial Sequence
  Mutated Ypep sequence
YTFSLKTSFNVQ

SEQ ID NO: 19 - protein, 12 aa, Artificial Sequence
  Mutated Ypep sequence
YTFNLKTSFNVQ

SEQ ID NO: 20 - protein, 12 aa, Artificial Sequence
  Mutate Ypep sequence
YTFQLKTSFNVQ

SEQ ID NO: 21 - protein, 12 aa, Artificial Sequence
  Mutated Ypep sequence
YTFGLKASFNVQ

SEQ ID NO: 22 - protein, 12 aa, Artificial Sequence
  Mutated Ypep sequence
YTFGLKDSFNVQ

SEQ ID NO: 23 - protein, 12 aa, Artificial Sequence
  Mutated Ypep sequence
YTFGLKKSFNVQ

SEQ ID NO: 24 - protein, 12 aa, Artificial Sequence
  Mutated Ypep sequence
YTFGLKSSFNVQ

SEQ ID NO: 25 - protein, 12 aa, Artificial Sequence
  Mutated Ypep sequence
YTFGLKNSFNVQ

SEQ ID NO: 26 - protein, 12 aa, Artificial Sequence
  Mutated Ypep sequence
YTFGLKFSFNVQ

SEQ ID NO: 27 - protein, 12 aa, Artificial Sequence
  Mutated Ypep sequence
YTFGLKYSFNVQ

SEQ ID NO: 28 - protein, 12 aa, Artificial Sequence
  Mutated Ypep sequence
YTFGLKWSFNVQ

SEQ ID NO: 29 - protein, 12 aa, Artificial Sequence
  Mutated Ypep sequence
YTFGLKHSFNVQ

SEQ ID NO: 30 - protein, 12 aa, Artificial Sequence
  Mutate Ypep sequence
YTFALKFSFNVQ

SEQ ID NO: 31 - protein, 12 aa, Artificial Sequence
  Mutated Ypep sequence
YTFKLKFSFNVQ

SEQ ID NO: 32 - protein, 12 aa, Artificial Sequence
  Mutated Ypep sequence
YTFNLKFSFNVQ